(19) United States
(12) Patent Application Publication
(10) Pub. No.: US 2024/0247264 A1
(43) Pub. Date: Jul. 25, 2024

(54) NOVEL lncRNA CONTROLLING CARDIAC FIBROSIS

(71) Applicant: CENTRE HOSPITALIER UNIVERSITAIRE VAUDOIS (C.H.U.V.), Lausanne (CH)

(72) Inventors: Thierry PEDRAZZINI, Le Mont-sur-Lausanne (CH); Parisa AGHAGOLZADEH, Basel (CH)

(21) Appl. No.: 18/564,201

(22) PCT Filed: May 25, 2022
(86) PCT No.: PCT/EP2022/064248

§ 371 Date: Nov. 27, 2023

(30) Foreign Application Priority Data
May 28, 2021 (EP) 21176732.2

(51) Int. Cl. C12N 15/113; A61P 9/00; C12Q 1/6883

(52) U.S. Cl. CPC C12N 15/113; A61P 9/00; C12Q 1/6883; C12N 2310/11; C12N 2310/321; C12N 2310/3231; C12N 2310/341; C12Q 2600/158

(57) ABSTRACT

This invention generally relates to methods for treating and/or preventing a cardiac pathology in a subject, comprising administering to said subject an effective amount of a modulator of FIXER, a novel lncRNA controlling cardiac fibrosis and remodeling following injury in the heart. The invention also provides methods for diagnosing cardiac pathologies in a subject based on FIXER expression in cardiac tissues or in body fluids.

### FIELD OF THE INVENTION

This invention generally relates to methods for treating a cardiac pathology in a subject, comprising administering to said subject an effective amount of a modulator of FIXER, a novel lncRNA controlling cardiac fibrosis and remodeling following injury in the heart. The invention also provides methods for diagnosing cardiac pathologies in a subject based on FIXER expression in cardiac tissues or in body fluids.

### SEQUENCE LISTING

The instant application contains a Sequence Listing, named PAT7579PC00_PCT_ST25.txt, which has been submitted electronically and is hereby incorporated by reference in its entirety.

### BACKGROUND OF THE INVENTION

Cardiovascular disease is the leading cause of death globally. Fibrotic scars in the cardiac muscle most commonly occur after myocardial infarction. However, there are various other conditions associated with the development of cardiac fibrosis, for instance but not restricted to, ischemic cardiomyopathies, hypertensive cardiomyopathies, diabetic cardiomyopathies, hereditary cardiomyopathies, myocarditis (incl. COVID-19), cancer therapy-induced cardiomyopathies, and ageing.

The fibrotic component in the diseased heart is very deleterious, contributes therefore to deterioration of heart function, and ultimately leads to heart failure, a condition characterized with very poor outcome. In this vein, reduction of fibrosis in the damaged myocardium, even modest, results in a significant therapeutic benefit.

Drugs used to treat cardiac patients are aimed mainly at preserving residual cardiac function following injury. In this context, no treatment is currently available for directly targeting fibrosis in the heart. In pre-clinical studies, different approaches to modulate specifically the fibrotic response in the heart have been evaluated, which support the beneficial effects of direct anti-fibrotic treatments. Thus, the field is in need of therapeutic molecules to precisely control the development of fibosis in the injured heart.

The development of cardiac fibrosis is controlled by the spatiotemporal execution of evolutionary conserved cardiac gene regulatory networks (GRNs). GRNs themselves consist of interacting and interleaved protein-coding and non protein-coding RNA networks. Non-coding RNAs include small non-coding RNA species (e.g. miRNAs) and, particularly, long non-coding RNAs (lncRNAs), which have been identified as important regulatory molecules within GRNs. LncRNAs have many distinct regulatory functions including the targeting of chromatin remodelling complexes to specific genomic loci, repression and enhancement of coding and non-coding gene expression and post-transcriptional modulation of protein coding and non-coding RNAs. This allows the establishment of cell-specific epigenomic states that tighly control cell identity and behavior. Thus, cardiac cell-enriched lncRNAs are critical regulators within cardiac GRNs, being thereby key determinants of pathological cardiac remodelling after injury. Identification and characterization of cardiac fibroblast- and myofibroblast-specific lncRNAs provide ways of controlling matrix-protein expression and the development of fibrosis in the heart.

### SUMMARY OF THE INVENTION

The invention relates to a method for diagnosing a cardiac pathology in a subject, the method comprising:
a) measuring, directly or indirectly, the level of at least one transcript encoded by a DNA sequence comprising the sequence of SEQ ID No. 1, a fragment thereof, and a variant sharing at least 80% nucleotide sequence identity thereto, in a biological sample of said subject,
b) analyzing the level of said at least one transcript, a fragment thereof, an isoform or variant thereof, in conjunction with respective reference value ranges for said transcript,
wherein the transcript is a lncRNA, and
wherein differential expression of said lncRNA, fragment, isoform or variant thereof, in the biological sample compared to a control sample from a normal subject indicates that the subject has a cardiac pathology.

A further object of the present invention is to provide an agent (e.g. modulator) modulating the expression and/or activity
i) of at least one lncRNA, a fragment thereof, an isoform thereof and a variant sharing at least 80% nucleotide sequence identity thereto, encoded by a DNA sequence comprising the sequence of SEQ ID No. 1, a fragment thereof, and a variant sharing at least 80% nucleotide sequence identity thereto, or
ii) of a DNA sequence comprising the sequence of SEQ ID No. 1, a fragment thereof, and a variant sharing at least 80% nucleotide sequence identity thereto, wherein said DNA sequence encodes at least one lncRNA, a fragment thereof, an isoform thereof and a variant sharing at least 80% nucleotide sequence identity thereto.

A further object of the present invention is to provide a vector comprising one or more nucleic acid of the invention.

A further object of the invention is to provide a host cell comprising, or modified by the introduction of, i) one or more nucleic acid of the invention, or ii) the vector of the invention.

Also provided is a pharmaceutical composition comprising a therapeutically effective amount of i) an agent of the invention, ii) a vector of the invention, or iii) a host cell of the invention, in combination with pharmaceutically acceptable carriers, diluents and/or adjuvants.

Also provided is at least one lncRNA, fragment, isoform or variant thereof encoded by a DNA sequence comprising the sequence of SEQ ID No. 1, a fragment thereof, and a variant sharing at least 80% nucleotide sequence identity thereto.

Also provided is a kit comprising
i) a composition comprising the lncRNA modulator wherein the modulator is selected from the group comprising a chemical agent, a RNA mimic, an antibody, an engineered protease, and enzymatically active RNA or
ii) a pharmaceutical composition comprising an effective amount of the lncRNA modulator wherein the modulator is selected from the group comprising a chemical agent, a RNA mimic, an antibody, an engineered protease, and enzymatically active RNA, optionally in combination with pharmaceutically acceptable carriers, diluents and/or adjuvants.

Also provided is a diagnostic kit comprising
i) one or more agents for detection of at least one lncRNA, a fragment thereof, an isoform thereof and a variant sharing at least 80% nucleotide sequence identity thereto, encoded by a DNA sequence comprising the sequence of SEQ ID No. 1, a fragment thereof, and a variant sharing at least 80% nucleotide sequence identity thereto,
ii) a container for holding a biological sample isolated from a human subject; and
iii) printed instructions.

Further provided is method of treating and/or preventing a cardiac pathology in a subject in need thereof, comprising administering to said subject a therapeutically effective amount of i) an agent of the invention, or ii) of a pharmaceutical composition of the invention.

### DESCRIPTION OF THE FIGURES

FIG. 1. LncRNA profiling identifies relevant cardiac cell types in single-cell RNA sequencing analysis A) Schematic of cardiac non-myocyte cell isolation from sham-operated and infarcted (MI) hearts. Surgeries were performed in mice at 10 weeks of age (P70). Cells were isolated 3 days post-surgery (n=4). B) Purification of cardiac DAPI-Calcein⁺ CD45⁻ CD31⁻ non-myocyte cells by FACS for single-cell RNA analysis. Single-cell RNA analysis was performed using a 10× Genomics platform. C) Clustering and visualization (t-SNE plot) of non-myocyte cells based exclusively on lncRNA expression. Cell types are identified based on expression of markers as depicted in D. D) t-SNE plots showing expression of cell type-specific markers for each cluster. E) t-SNE plot highlighting clusters only present in the infarcted heart (surrounded by a dashed line). F) Percentage of cells in each cluster/subpopulation in the sham-operated vs. the infarcted heart. G-H) Heatmaps showing the expression of cluster/subpopulation-specific noncoding (G) and coding transcripts (H).

FIG. 2. Fixer is a novel cardiac fibroblast-enriched lncRNA, highly upregulated in the heart after MI. A-B) Re-clustering of cardiac fibroblasts (Clusters A and D as indicated in FIG. 1C) reveals population heterogeneity and identifies infarction-specific fibroblast subpopulations (surrounded by a dashed line in B). C) t-SNE plots showing expression of cell type-specific markers for each cluster. Expression of Col1a1, Col3a1, Fn1, Postn and Acta2 identifies myofibroblasts. D) Bioinformatics pipeline used to identify FIXER, a lncRNA expressed in myofibroblasts after MI.

FIG. 3. FIXER is expressed in cardiac fibroblasts differentiating into myofibroblasts in vitro and in vivo. A) Schematic of cardiac non-myocyte cell isolation at different times after MI in the mouse. B) Expression kinetics of FIXER and the adjacent gene Lox in the heart after MI. C) Expression kinetics of Myofibroblast markers (Col1a1, Col3a1, Postn and Acta2) in the heart after MI. D) FIXER and Lox expression measured in the major cardiac cell populations before and after MI in neonatal (P1) and adult mice (P56). Data represent transcript levels as measured by RNA sequencing. E) Reduced expression of FIXER, Lox and myofibroblast markers in cardiac fibroblasts following GapmeR-mediated FIXER silencing (SEQ ID No.15). Scrambled GapmeRs are used as control (25 nM, 48 hours; n=3). F) LOX activity in cardiac fibroblasts measured following GapmeR-mediated FIXER silencing in the presence or absence of TGF-β (5 nM, 48 hours; n≥4). Mean±SEM; *p<0.05, **p<0.01.

FIG. 4. Fixer silencing reduced cardiac fibrosis and remodeling in the infarcted heart. A) Overview of the experimental setup. GapmeRs targeting Fixer or scrambled GapmeRs (SEQ ID No. 15 and No. 16; 10 mg/kg) were injected at 2, 4 and 9 days post-infarction in the mouse (n≥9). Assessment was performed 14 days after MI. B-C) Expression of Fixer and markers of fibrosis and remodeling in treated sham-operated and infarcted mice 14-days after surgery (light gray: Scrambled GapmeRs; dark gray: Fixer GapmeRs). D) Representative Masson's trichrome staining of heart sections in treated sham-operated and infarcted mice 14-days after surgery (Darker area represent fibrotic tissue), and quantification. E) Infarcted size as measured by echocardiography in treated sham-operated and infarcted mice 14-days after surgery (n≥5). F) Echocardiographic assessment of cardiac dimension (IVS d, diastolic intraventricular septum; LVPW d, diastolic left ventricular posterior wall thickness; LVID s, systolic left ventricular internal diameter) in treated sham-operated and infarcted mice 14-days after surgery (n≥9). G) Representative M-mode images of the left ventricle, and echocardiographic assessment of heart function in treated sham-operated and infarcted mice 14-days after surgery (% EF, Ejection fraction, n≥9). Means±SEM. P values were determined by one-way ANOVA, light gray: Scrambled GapmeRs; dark gray: Fixer GapmeRs).

FIG. 5. FIXER controls the fibroblast gene program A) Principal component analysis (PCA) of RNA samples isolated from sham-operated and infarcted hearts treated with scrambled GapmeRs or GapmeRs targeting FIXER (10 mg/kg; n≥3). B) Protein-coding genes (PCGs) differentially modulated in Sham-operated heart treated with Scrambled vs. FIXER GapmeRs and in infarcted heart treated with Scrambled vs. FIXER GapmeRs. Venn diagram shows that 209 and 1734 PCGs are modulated specifically in the sham-operated group and the infracted group respectively. C) Heatmap illustrates the expression patterns of the 1734 differentially expressed PCGs (1399 upregulated; 335 downregulated) in the sham-operated and the infarcted group before and after FIXER GapmeR treatment. The expression of the upregulated PCGs after infarction is blunted by the anti-FIXER treatment. D) Gene ontology terms associated to the upregulated genes after infarction. The Circus plot represents the association between the top PCGs sensitive to anti-Fixer treatment and biological pathways relevant to cardiac fibrosis and remodeling. E) Heatmap illustrates the expression patterns of 306 up- and 374 downregulated lncRNAs in the sham-operated and the infarcted group before and after FIXER GapmeR treatment. The expression of the upregulated lncRNAs after infarction is blunted by the anti-FIXER treatment. F) Kernel density plot displaying correlation between differentially modulated lncRNAs and their respective adjacent PCGs, and between differentially modulated lncRNAs and a randomly selected set of PCGs, LncRNA expression is better correlated with adjacent gene expression.

FIG. 6. FIXER is conserved in human. A) UCSC genome browser views of mouse FIXER, depicting also sequence conservation in human. Conserved sequences were used to predict position of exons in human FIXER. PCR primers used to amplify regions corresponding to human exons are indicated as gray boxes. Primer pairs 1 and 2 and primer pairs 3 and 4 are used to amplify the indicated conserved regions in PCR followed by sequencing (B-C) and in quantitative RT-PCR (D-E). B-C) Sequences of the amplicons produced by PCR using primer pairs 1 and 3, and RNA samples isolated from the human heart. D-E) Measurement of FIXER expression in the human heart using quantitative RT-PCR. RNA samples were isolated from the right and left ventricles (RV; LV), and from cultured human cardiac fibroblasts (CF) using primer pairs 2 and 4. F) Measurement of FIXER expression in human cardiac fibroblasts isolated from the normal heart following exposure to TGFβ using primer pairs 2. FIXER expression is downregulated upon administration of anti-FIXER GapmeRs together with typical fibroblastic genes. G) Measurement of FIXER expression in human cardiac fibroblasts isolated from the heart of aortic stenosis patients using primer pairs 2. FIXER expression is downregulated upon administration of anti-FIXER GapmeRs together with typical fibroblastic genes. H) Measurement of FIXER expression in the fibrotic heart of aortic stenosis patients using primer pairs 2. I) Measurement of FIXER expression in the fibrotic heart of aortic stenosis patients using primer pairs 2. FIXER expression correlates with cardiac insoluble collagen content and with cardiac collagen crosslinking.

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms “comprise(s)” and “comprising” also encompass the more restricted ones “consist(s)”, “consisting” as well as “consist/consisting essentially of”, respectively.

As used in the specification and claims, the singular form “a”, “an” and “the” include plural references unless the context clearly dictates otherwise.

As used herein the terms “subject”/“subject in need thereof”, or “patient”/“patient in need thereof” are well-recognized in the art, and, are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease or disorder. However, in other aspects, the subject can be a normal subject. The term does not denote a particular age or sex. Thus, adult and newborn subjects, whether male or female, are intended to be covered. Preferably, the subject is a human, most preferably a human suffering from a cardiac disease or a human that might be at risk of suffering from a cardiac disease.

As disclosed herein, the “cardiac disease” is usually selected from the non-limiting group comprising Blood vessel disease, such as coronary artery disease; Stenosis, such as aortic stenosis; Myocardial infarction; Heart failure, such as heart failure with preserved ejection fraction, heart failure with reduced ejection fraction, and heart failure with mid-range ejection fraction; Heart rhythm problems, such as arrhythmias; Cardiac fibrosis; Heart defects, such as congenital heart defects; Heart valve disease; Genetic heart disease; Cardiomyopathy such as idiopathic and dilated cardiomyopathy; Diabetic cardiomyopathy; Toxic and drug-induced cardiomyopathy; Heart infection, such as myocarditis (incl. COVID-19); Rheumatic Heart Disease; Hypertension, such as essential and renovascular hypertension; Pulmonary hypertension; Muscular dystrophy; Cardiac pathologies associated to neurological disorders; Cardiac pathologies associated to cancer; Traumatic heart disease; and pericardial disease, affecting or not the function of the atria or the cardiac ventricles of the right or left heart, ischemic cardiomyopathies, hypertensive cardiomyopathies, diabetic cardiomyopathies, hereditary cardiomyopathies, cancer therapy-induced cardiomyopathies, and ageing or a combination of one or more thereof.

The terms “nucleic acid”, “polynucleotide,” and “oligonucleotide” are used interchangeably and refer to any kind of deoxyribonucleotide (e.g. DNA, cDNA, . . . ) or ribonucleotide (e.g. RNA, mRNA, ncRNA, . . . ) polymer or a combination of deoxyribonucleotide and ribonucleotide (e.g. DNA/RNA) polymer, in linear or circular conformation, and in either single- or double-stranded form. These terms are not to be construed as limiting with respect to the length of a polymer and can encompass known analogues of natural nucleotides, as well as nucleotides that are modified in the base, sugar and/or phosphate moieties (e.g. phosphorothioate backbones). In general, an analogue of a particular nucleotide has the same base-pairing specificity, i.e., an analogue of A will base-pair with T. Also included are polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, microRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30: 1911-1918 incorporated by reference in its entirety.

The term “vector”, as used herein, refers to a viral vector or to a nucleic acid (DNA or RNA) molecule such as a plasmid or other vehicle, which contains one or more heterologous nucleic acid sequence(s) and is designed for transfer between different host cells. The terms “expression vector”, “gene delivery vector” and “gene therapy vector” refer to any vector that is effective to incorporate and express one or more nucleic acid(s), in a cell, preferably under the regulation of a promoter. A cloning or expression vector may comprise additional elements, for example, regulatory and/or post-transcriptional regulatory elements in addition to a promoter.

The term “about” particularly in reference to a given quantity, is meant to encompass deviations of plus or minus ten percent (±10%). For example, “at least 80%” also encompasses a value between 72% and 88%.

The term “treatment” or “treating” means any administration of a composition, pharmaceutical composition, therapeutic agent, compound, etc. . . . of the disclosure to a subject for the purpose of:
(i) inhibiting the disease, that is, arresting the development of clinical symptoms; and/or
(ii) relieving the disease, that is, causing the regression of clinical symptoms.

As used herein, the term “prevention” or “preventing” means any administration of a composition, pharmaceutical composition, therapeutic agent, compound, etc. . . . of the disclosure to a subject for the purpose of:
(i) preventing the disease, that is, causing the clinical symptoms of the disease not to develop.

In the context of the present invention, the disease is a cardiac disease as disclosed herein.

In an aspect of the invention, the terms “agent modulating”, “agent of the invention” and “modulator” are used interchangeably.

While focusing on developing a novel functional annotation of the lnRNA transcriptome in cardiac fibroblasts the Inventors surprisingly characterized fibroblasts and myofibroblasts associated lncRNA transcriptome.

One of them, the CF-enriched lncRNA, which is highly upregulated in the heart after a cardiac disease, such as e.g. myocardial infarction, is a lncRNA which gene is located adjacent to the Lox gene encoding a lysyl oxidase implicated in collagen crosslinking and has been named FIXER (Fibroblast-enriched Lox-locus enhancer-RNA).

TABLE 1

| lncRNA primer | DNA sequence | SEQ ID No. |
|---|---|---|
| Primer 1 Forward | CTTGCATGCCTTCCTAAACTC | 2 |
| Primer 1 Reverse | AACATTGTTATCAATCAAGATTCAGTT | 3 |
| Primer 2 Forward | CATGCCTTCCTAAACTCCAG | 4 |
| Primer 2 Reverse | ATTCCCGCATAATCATGCTT | 5 |
| Primer 3 Forward | TTGATATACAAAGAAAAGTTGGGAAT | 6 |
| Primer 3 Reverse | GGAGGCTCCTCATCATCTTG | 7 |
| Primer 4 Forward | CTGCTGAGATACAACCCACT | 8 |
| Primer 4 Reverse | TCCATCAGTCGTATCCCATATTG | 9 |
| Primer 5 Forward | GATGCAGCTTAAGGGTCTC | 10 |
| Primer 5 Reverse | TTAGGTTCACGGTCAGGTC | 11 |

Primers 1, 2, 3, and 4 (Forward and Reverse) are human primers whereas primers 5 (Forward and Reverse) are mouse primers.

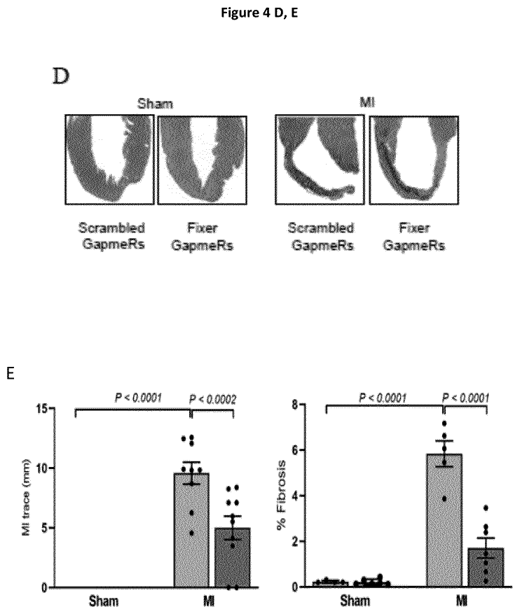

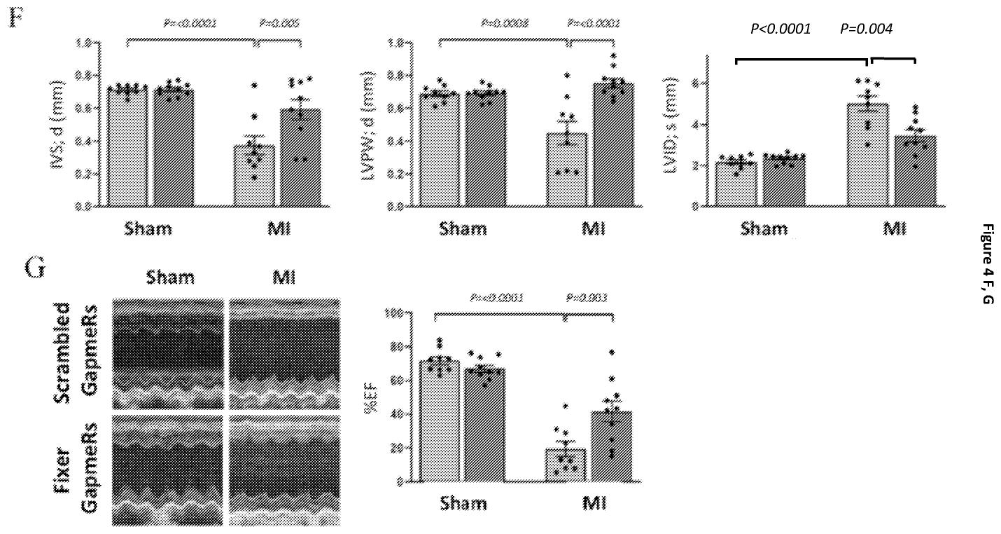

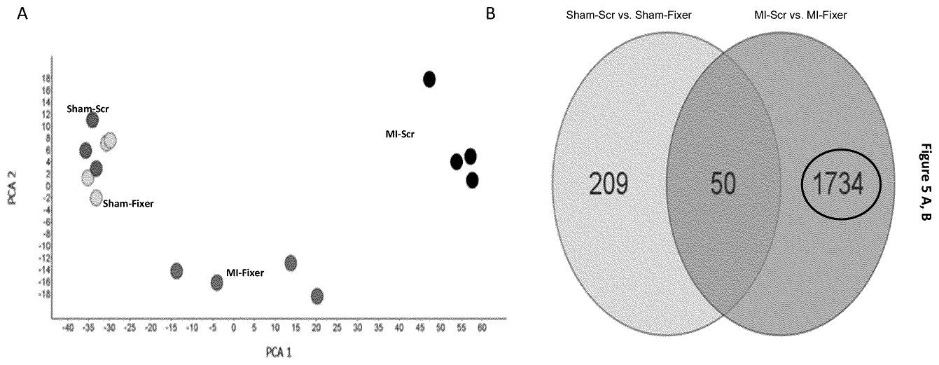

The inventors have shown that FIXER silencing reduced infarcted size and interstitial fibrosis (FIGS. 4D and E), diminished cardiac remodeling as assessed by echocardiography (FIG. 4F; IVS: intraventricular septum; LVPW: Left ventricular posterior wall; LVID: Left ventricular internal diameter), and significantly improved cardiac function (FIG. 4G; EF: Ejection fraction).

The present invention concerns a method for diagnosing a cardiac pathology in a subject, the method comprising:
a) measuring, directly or indirectly, the level of at least one transcript encoded by a DNA sequence comprising the sequence of SEQ ID No. 1, a fragment thereof, and a variant sharing at least 80% nucleotide sequence identity thereto, in a biological sample of said subject,
b) analyzing the level of said at least one transcript, a fragment thereof, an isoform or variant sharing at least 80% nucleotide sequence identity thereto, in conjunction with respective reference value ranges for said transcript,
wherein the transcript is a lncRNA, and
wherein differential expression of said lncRNA, fragment, isoform or variant thereof, in the biological sample compared to a control sample from a normal subject indicates that the subject has a cardiac pathology.

The phrase “differential expression” refers to differences in the quantity and/or the frequency of said lncRNA, fragment, isoform or variant thereof present in a sample taken from patients having, for example, a cardiac pathology or from a cardiac tissue undergoing regeneration or from a stem cell undergoing cardiac differentiation or from a cardiac tissue undergoing surgical and/or pharmacological therapies as compared to a control subject. In the context of the present invention, said lncRNA, fragment, isoform or variant thereof is present at an elevated level in samples of patients with a cardiac pathology or a cardiac tissue undergoing regeneration or a stem cell undergoing cardiac differentiation or a cardiac tissue undergoing surgical and/or pharmacological therapies compared to samples of control subjects. Alternatively, said lncRNA, fragment, isoform or variant thereof is detected at a higher frequency in samples of patients with a cardiac pathology or a cardiac tissue undergoing regeneration or a stem cell undergoing cardiac differentiation or a cardiac tissue undergoing surgical and/or pharmacological therapies compared to samples of control subjects or control tissues. The lncRNA, fragment, isoform or variant thereof can be differentially present in terms of quantity, frequency or both.

The lncRNA, fragment, isoform or variant thereof is differentially expressed between two samples if the amount of said lncRNA, fragment, isoform or variant thereof in one sample is statistically significantly different from the amount of the lncRNA, fragment, isoform or variant thereof in the other sample. For example, a lncRNA, fragment, isoform or variant thereof is differentially expressed in two samples if it is present at least about 50%, at least about 120%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, said lncRNA, fragment, isoform or variant thereof is differentially expressed in two sets of samples if the frequency of detecting the lncRNA, fragment, isoform or variant thereof in samples is statistically significantly higher than in the control samples. For example, said lncRNA, fragment, isoform or variant thereof is differentially expressed in two sets of samples if it is detected at least about 50%, at least about 120%, at least about 150%, at least about 180%, at least about 200%, at least about 300%, at least about 500%, at least about 700%, at least about 900%, or at least about 1000% more frequently observed in one set of samples than the other set of samples.

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the lncRNA, fragment, isoform or variant thereof of the invention. These values or ranges can be obtained from a single patient or from a group of patients.

The terms “variant” refers to one or more biologically active derivatives of a lncRNA or a DNA sequence of the invention. In general, the term “variant” refers to molecules having a native sequence and structure with one or more additions, substitutions (generally conservative in nature) and/or deletions, relative to the native molecule, so long as the modifications do not destroy its biological activity and which are “substantially homologous” to the reference molecule or SEQ ID. In general, the sequences of such variants will have a high degree of sequence homology to the reference sequence, e.g., sequence homology of more than 50%, generally more than 60% to 70%, even more particularly 80%, or 85% or more, such as at least 90%, or 95% or more, when the two sequences are aligned.

In one aspect, the term “variant” also refers to post-transcriptionally modified lncRNA of the invention, i.e methylation, phosphorylation, polyadenylation, etc . . . .

In another aspect of the invention, the term “variant” also comprises precursors of the lncRNA transcript and splicing variants thereof. Alternative splicing is a widely used mechanism for the formation of isoforms or splicing variants. In this process, which occurs during gene expression, the exons of a gene may be included or excluded in the processed RNA.

As used, “Homology” refers to the percent identity between two polynucleotide or two polypeptide moieties. Homology can be determined by readily available computer programs or by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

Alternatively, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100.

As used herein, a “fragment” of a lncRNA or DNA of the invention refers to a sequence containing less nucleotides in length than the respective lncRNA sequence or DNA sequence. Preferably, this sequence contains less than 90%, preferably less than 60%, in particular less than 30% nucleotides in length than the respective lncRNA sequence or DNA sequence.

In an aspect of the invention, the level of the lncRNA, fragment, isoform or variant thereof, is detected using a method selected from the group comprising RNA sequencing, microarray analysis, polymerase chain reaction (PCR), reverse transcription polymerase chain reaction (RT-PCR), dual-labeled probe method, Northern blot, serial analysis of gene expression (SAGE), immunoassay, and mass spectrometry, or a combination of one or more thereof.

In an aspect of the invention, the cardiac pathology is selected from the group comprising Blood vessel disease, such as coronary artery disease; Stenosis, such as aortic stenosis; Myocardial infarction; Heart failure, such as heart failure with preserved ejection fraction, heart failure with reduced ejection fraction, and heart failure with mid-range ejection fraction; Heart rhythm problems, such as arrhythmias; Cardiac fibrosis; Heart defects, such as congenital heart defects; Heart valve disease; Genetic heart disease; Cardiomyopathy such as idiopathic and dilated cardiomyopathy; Diabetic cardiomyopathy; Toxic and drug-induced cardiomyopathy; Heart infection, such as myocarditis (incl. COVID-19); Rheumatic Heart Disease; Hypertension, such as essential and renovascular hypertension; Pulmonary hypertension; Muscular dystrophy; Cardiac pathologies associated to neurological disorders; Cardiac pathologies associated to cancer; Traumatic heart disease; and pericardial disease, affecting or not the function of the atria or the cardiac ventricles of the right or left heart. ischemic cardiomyopathies, hypertensive cardiomyopathies, diabetic cardiomyopathies, hereditary cardiomyopathies, cancer therapy-induced cardiomyopathies, and ageing or a combination of one or more thereof. In a preferred aspect, the cardiac pathology is Myocardial infarction (MI).

Usually, the biological sample is selected from the group comprising whole blood, serum, plasma, semen, saliva, tears, urine, fecal material, sweat, buccal and nasal smears, amniotic fluid, tissue sample, biopsy and hair, or a combination of one or more thereof.

In an aspect of the invention, the method for diagnosing a cardiac pathology in a subject further comprises a step (step c) of administering to the subject diagnosed as having, or suffering from a cardiac pathology, an agent (e.g. modulator) modulating the expression and/or activity
i) of at least one lncRNA, a fragment thereof, an isoform thereof and a variant sharing at least 80% nucleotide sequence identity thereto, encoded by a DNA sequence comprising the sequence of SEQ ID No. 1, a fragment thereof, and a variant sharing at least 80% nucleotide sequence identity thereto, or
ii) of a DNA sequence comprising the sequence of SEQ ID No. 1, a fragment thereof, and a variant sharing at least 80% nucleotide sequence identity thereto, wherein said DNA sequence encodes at least one lncRNA, a fragment thereof, an isoform thereof and a variant sharing at least 80% nucleotide sequence identity thereto, or
iii) any other cardiac pathology treatment alone or in combination with an agent described herein, or a combination of i), ii) and/or iii).

It is understood that the expression level of the lncRNA, fragment, isoform or variant thereof in a sample can be determined by any suitable method known in the art. Measurement of the level of a lncRNA of the invention, fragment, isoform or variant thereof can be direct or indirect. For example, the abundance levels of lncRNAs can be directly quantitated. Alternatively, the amount of a lncRNA, fragment, isoform or variant thereof can be determined indirectly by measuring abundance levels of cDNAs, amplified RNAs or DNAs, or by measuring quantities or activities of RNAs, or other molecules that are indicative of the expression level of the lncRNA, fragment, isoform or variant thereof. Preferably, the amount of a lncRNA, fragment, isoform or variant thereof is determined indirectly by measuring abundance levels of DNAs or cDNAs.

A lncRNA can be detected and quantitated by a variety of methods including, but not limited to, RNA sequencing, microarray analysis, polymerase chain reaction (PCR), reverse transcription polymerase chain reaction (RT-PCR), dual-labeled probe method, Northern blot, serial analysis of gene expression (SAGE), immunoassay, and mass spectrometry, and any sequencing-based methods known in the art.

In one aspect, microarrays are used to measure the levels of lncRNA, fragment, isoform or variant thereof. An advantage of microarray analysis is that the expression of each of the lncRNA, fragment, isoform or variant thereof can be measured simultaneously, and microarrays can be specifically designed to provide a diagnostic expression profile for a particular disease or condition (e.g., a cardiac pathology).

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes used in the methods of the invention are preferably immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well known in the art (see, e.g., Sambrook, et al., Molecular Cloning: A Laboratory Manual (3rd Edition, 2001). Alternatively, the solid support or surface may be a glass or plastic surface. In one embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

Any probe that selectively hybridizes to a least one lncRNA of the invention, fragment, isoform or variant thereof, is also encompassed in the present invention.

In one aspect, the microarray comprises a support or surface with an ordered array of binding (e.g., hybridization) sites or “probes” wherein at least one represents the lncRNA described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site.

Microarrays can be made in a number of ways, of which several are described below. However, they are produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. Microarrays are generally small, e.g., between 1 cm2 and 25 cm2; however, larger arrays may also be used, e.g., in screening arrays. Preferably, a given binding site or unique set of binding sites in the microarray will specifically bind (e.g., hybridize) to the product of a single gene in a cell (e.g., to a specific mRNA, lncRNA, or to a specific cDNA derived therefrom). However, in general, other related or similar sequences will cross hybridize to a given binding site.

As noted above, the “probe” to which a particular polynucleotide molecule specifically hybridizes contains a complementary polynucleotide sequence. The probes of the microarray typically consist of nucleotide sequences of no more than 1,000 nucleotides. In some embodiments, the probes of the array consist of nucleotide sequences of 10 to 1,000 nucleotides. In one embodiment, the nucleotide sequences of the probes are in the range of 10-200 nucleotides in length and are genomic sequences of one species of organism, such that a plurality of different probes is present, with sequences complementary and thus capable of hybridizing to the genome of such a species of organism, sequentially tiled across all or a portion of the genome. In other embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, or are 60 nucleotides in length. The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone (e.g., phosphorothioates).

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically, each probe on the microarray will be between 10 bases and 50,000 bases, usually between 300 bases and 1,000 bases in length. PCR methods are well known in the art, and are described, for example, in Innis et al., eds., PCR Protocols: A Guide To Methods And Applications, Academic Press Inc., San Diego, Calif. (1990); herein incorporated by reference in its entirety. It will be apparent to one skilled in the art that controlled robotic systems are useful for isolating and amplifying nucleic acids.

Any primer that provides specificity and optimal amplification properties of a least one lncRNA, fragment, isoform or variant thereof, of the invention is encompassed.

An alternative, preferred means for generating polynucleotide probes is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is a peptide nucleic acid (see, e.g., U.S. Pat. No. 5,539,083).

Probes are preferably selected using an algorithm that takes into account binding energies, base composition, sequence complexity, cross-hybridization binding energies, and secondary structure.

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. One method for attaching nucleic acids to a surface is by printing on glass plates, as known in the art. This method is especially useful for preparing microarrays of cDNA A second method for making microarrays produces high-density oligonucleotide arrays. Techniques are known for producing arrays containing thousands of oligonucleotides complementary to defined sequences, at defined locations on a surface using photolithographic techniques for synthesis in situ (see, U.S. Pat. Nos. 5,578,832; 5,556,752; and 5,510,270; herein incorporated by reference in their entireties) or other methods for rapid synthesis and deposition of defined oligonucleotides. When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking, may also be used. In principle, any type of array known in the art, for example, dot blots on a nylon hybridization membrane could be used. However, as will be recognized by those skilled in the art, very small arrays will frequently be preferred because hybridization volumes will be smaller.

Microarrays can also be manufactured by means of an ink jet printing device for oligonucleotide synthesis, e.g., using the methods and systems described by Blanchard in U.S. Pat. No. 6,028,189. Specifically, the oligonucleotide probes in such microarrays are synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink jet method are typically of high density, preferably having a density of at least about 2,500 different probes per 1 cm2. The polynucleotide probes are attached to the support covalently at either the 3′ or the 5′ end of the polynucleotide.

The lncRNA, fragment, isoform or variant thereof polynucleotides which may be measured by microarray analysis can be expressed lncRNAs or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one aspect, the target polynucleotide molecules comprise RNA, including, but by no means limited to, total cellular RNA, lncRNA, poly(A)+ messenger RNA (mRNA) or a fraction thereof, cytoplasmic mRNA, or RNA transcribed from cDNA (i.e., cRNA; see, e.g., U.S. Pat. Nos. 5,545,522, 5,891,636, or 5,716,785). Methods for preparing total and poly(A)+ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation, a silica gel-based column (e.g., RNeasy (Qiagen, Valencia, Calif.) or StrataPrep (Stratagene, La Jolla, Calif.)), or using phenol and chloroform, as known in the art. Poly(A)+ RNA can be selected, e.g., by selection with oligo-dT cellulose or, alternatively, by oligo-dT primed reverse transcription of total cellular RNA. RNA can be fragmented by methods known in the art, e.g., by incubation with ZnCl2, to generate fragments of RNA.

In one aspect, total RNA, lncRNAs, or nucleic acids derived therefrom (such as cDNA), are isolated from a sample taken from a patient having a cardiac pathology or a cardiac tissue undergoing regeneration or a stem cell undergoing cardiac differentiation or a cardiac tissue undergoing surgical and/or pharmacological therapies. LncRNA, fragment, isoform or variant thereof that are poorly expressed in particular cells may be enriched using normalization techniques known in the art.

As described above, the biomarker polynucleotides can be detectably labeled at one or more nucleotides. Any method known in the art may be used to label the target polynucleotides. Preferably, this labeling incorporates the label uniformly along the length of the RNA, and more preferably, the labeling is carried out at a high degree of efficiency. For example, polynucleotides can be labeled by oligo-dT primed reverse transcription. Random primers (e.g., 9-mers) can be used in reverse transcription to uniformly incorporate labeled nucleotides over the full length of the polynucleotides. Alternatively, random primers may be used in conjunction with PCR methods or T7 promoter-based in vitro transcription methods in order to amplify polynucleotides.

The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. Alternatively, the detectable label can be a radiolabeled nucleotide.

In one aspect, lncRNA polynucleotide molecules from a patient sample are labeled differentially from the corresponding polynucleotide molecules of a reference sample. The reference can comprise lncRNAs from a normal biological sample (i.e., control sample, e.g., biopsy from a subject not having a cardiac pathology or a cardiac tissue undergoing regeneration or a stem cell undergoing cardiac differentiation or a cardiac tissue undergoing surgical and/or pharmacological therapies) or from a reference biological sample, (e.g., sample from a subject having a cardiac pathology or cell sample of a cardiac tissue undergoing regeneration or a stem cell undergoing cardiac differentiation or a cardiac tissue undergoing surgical and/or pharmacological therapies).

Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS). Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labeled gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously. Arrays can be scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are known in the art. Alternatively, a fiber-optic bundle, may be used to monitor mRNA abundance levels at a large number of sites simultaneously.

In one aspect, the invention includes a microarray comprising at least one probe that hybridizes, preferably specifically or selectively, to the LncRNA of the invention, a fragment, an isoform or a variant thereof.

In a further aspect, the invention includes at least one probe that hybridizes, preferably specifically or selectively, to the LncRNA of the invention, a fragment, an isoform or a variant thereof.

Serial Analysis Gene Expression (SAGE), can also be used to determine RNA (e.g., lncRNA) abundances in a cell sample. SAGE analysis does not require a special device for detection, and is one of the preferable analytical methods for simultaneously detecting the expression of a large number of transcription products. First, RNA is extracted from cells. Next, the RNA is converted into cDNA using a biotinylated oligo (dT) primer, and treated with a four-base recognizing restriction enzyme (Anchoring Enzyme: AE) resulting in AE-treated fragments containing a biotin group at their 3′ terminus. Next, the AE-treated fragments are incubated with streptoavidin for binding. The bound cDNA is divided into two fractions, and each fraction is then linked to a different double-stranded oligonucleotide adapter (linker) A or B. These linkers are composed of: (1) a protruding single strand portion having a sequence complementary to the sequence of the protruding portion formed by the action of the anchoring enzyme, (2) a 5′ nucleotide recognizing sequence of the IIS-type restriction enzyme (cleaves at a predetermined location no more than 20 by away from the recognition site) serving as a tagging enzyme (TE), and (3) an additional sequence of sufficient length for constructing a PCR-specific primer. The linker-linked cDNA is cleaved using the tagging enzyme, and only the linker-linked cDNA sequence portion remains, which is present in the form of a short-strand sequence tag. Next, pools of short-strand sequence tags from the two different types of linkers are linked to each other, followed by PCR amplification using primers specific to linkers A and B. As a result, the amplification product is obtained as a mixture comprising myriad sequences of two adjacent sequence tags (ditags) bound to linkers A and B. The amplification product is treated with the anchoring enzyme, and the free ditag portions are linked into strands in a standard linkage reaction. The amplification product is then cloned. Determination of the clone's nucleotide sequence can be used to obtain a read-out of consecutive ditags of constant length. The presence of mRNA corresponding to each tag can then be identified from the nucleotide sequence of the clone and information on the sequence tags.

Quantitative reverse transcriptase PCR (qRT-PCR) can also be used to determine the expression profiles of biomarkers (see, e.g., U.S. Patent Application Publication No. 2005/0048542A1; herein incorporated by reference in its entirety). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and beta-actin.

A variation of the RT-PCR technique is the real time quantitative PCR, which measures PCR product accumulation through a dual-labeled fluorigenic probe (i.e., TAQMAN probe). Real time PCR is compatible both with quantitative competitive PCR, where internal competitor for each target sequence is used for normalization, and with quantitative comparative PCR using a normalization gene contained within the sample, or a housekeeping gene for RT-PCR.

Mass spectrometry, and particularly SELDI mass spectrometry, is a particularly useful method for detection of the biomarkers of this invention. Laser desorption time-of-flight mass spectrometer can be used in embodiments of the invention. In laser desorption mass spectrometry, a substrate or a probe comprising biomarkers is introduced into an inlet system. The lncRNA, fragment, isoform or variant thereofs are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting the lncRNA, fragment, isoform or variant thereofs of this invention. MALDI-MS is a method of mass spectrometry that involves the use of an energy absorbing molecule, frequently called a matrix, for desorbing proteins intact from a probe surface. MALDI is described, for example, in U.S. Pat. Nos. 5,118,937 and 5,045,694. In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Surface-enhanced laser desorption/ionization mass spectrometry, or SELDI-MS represents an improvement over MALDI for the fractionation and detection of biomolecules, such as lncRNAs, in complex mixtures. SELDI is a method of mass spectrometry in which biomolecules, such as lncRNAs, are captured on the surface of a biochip using capture reagents that are bound there. Typically, non-bound molecules are washed from the probe surface before interrogation. SELDI is described, for example, in: U.S. Pat. No. 5,719,060 and in U.S. Pat. No. 6,225,047.

The lncRNA, fragment, isoform or variant thereof on the substrate surface can be desorbed and ionized using gas phase ion spectrometry. Any suitable gas phase ion spectrometer can be used as long as it allows lncRNA, fragment, isoform or variant thereof on the substrate to be resolved. Preferably, gas phase ion spectrometers allow quantitation of lncRNA, fragment, isoform or variant thereof. In one aspect, a gas phase ion spectrometer is a mass spectrometer. In a typical mass spectrometer, a substrate or a probe comprising lncRNA, fragment, isoform or variant thereof on its surface is introduced into an inlet system of the mass spectrometer. The lncRNA, fragment, isoform or variant thereof are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of lncRNA, fragment, isoform or variant thereof or other substances will typically involve detection of signal intensity. This, in turn, can reflect the quantity and character of lncRNA, fragment, isoform or variant thereof bound to the substrate. Any of the components of a mass spectrometer (e.g., a desorption source, a mass analyzer, a detector, etc.) can be combined with other suitable components described herein or others known in the art in embodiments of the invention.

The lncRNA of the invention, a fragment, an isoform or a variant thereof can also be detected with assays based on the use of antibodies that specifically recognize the lncRNA, fragment, isoform or variant thereof or polynucleotide or oligonucleotide fragments of the lncRNA, fragment, isoform or variant thereof. Such assays include, but are not limited to, immunohistochemistry (IHC), enzyme-linked immunosorbent assay (ELISA), radioimmunoassays (RIA), “sandwich” immunoassays, fluorescent immunoassays, immunoprecipitation assays, the procedures of which are well known in the art.

The lncRNA of the invention, a fragment, an isoform or a variant thereof can also be detected with any sequencing-based technologies known in the art.

Non-limiting examples of primers useful in detecting and quantitating the lncRNA of the invention, fragment, isoform or variant in methods described herein are selected among the group comprising a sequence as set forth in SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, and SEQ ID No. 11, or a combination of one or more thereof. Examples of combination of primers comprise a combination of SEQ ID No. 2 and SEQ ID No. 3, SEQ ID No. 4 and SEQ ID No. 5, SEQ ID No. 6 and SEQ ID No. 7, SEQ ID No. 8 and SEQ ID No. 9, as well as SEQ ID No. 10 and SEQ ID No. 11.

The present invention further provides an agent (e.g. modulator) modulating the expression and/or activity
i) of at least one lncRNA, a fragment thereof, an isoform thereof and a variant sharing at least 80% nucleotide sequence identity thereto, encoded by a DNA sequence comprising the sequence of SEQ ID No. 1, a fragment thereof, and a variant sharing at least 80% nucleotide sequence identity thereto, or
ii) of a DNA sequence comprising the sequence of SEQ ID No. 1, a fragment thereof, and a variant sharing at least 80% nucleotide sequence identity thereto, wherein said DNA sequence encodes at least one lncRNA, a fragment thereof, an isoform thereof and a variant sharing at least 80% nucleotide sequence identity thereto.

In a preferred aspect, the agent is for use in the treatment and/or prevention of a cardiac pathology in a subject in need thereof.

Preferably, the DNA sequence encoding the lncRNA, fragment, isoform and variant thereof, corresponds to a gene sequence comprising, or consisting of, SEQ ID No. 1, that is located on human chromosome 5 between positions 121425179-121444826.

In some aspects, the agent of the invention is selected from the group comprising a nucleic acid, a chemical compound, a peptide or analog thereof, an antibody or an antigen-binding fragment thereof, and an antibody mimetic, or a combination of one or more thereof.

In case the agent of the invention is a nucleic acid, then it will preferably be selected from the non-limiting group comprising a nucleic acid encoding a siRNA, a shRNA, a miRNA, a transfer RNA (tRNA), a piRNA, a heterogeneous nuclear RNA (hnRNA), an snRNA, an sgRNA used in a CRISPR-based loss- or gain-of-function system, an esiRNA, a single-stranded DNA, and an antisense oligonucleotide, or a fragment of one thereof or a combination of one or more thereof.

The terms “microRNA,” “miRNA,” and “MiR” are interchangeable and refer to endogenous or artificial non-coding RNAs that are capable of regulating gene expression. It is believed that miRNAs function via RNA interference.

The terms “siRNA” and “short interfering RNA” are interchangeable and refer to single-stranded or double-stranded RNA molecules that are capable of inducing RNA interference. SiRNA molecules typically have a duplex region that is between 18 and 30 base pairs in length.

The terms “piRNA” and “Piwi-interacting RNA” are interchangeable and refer to a class of small RNAs involved in gene silencing. PiRNA molecules typically are between about 26 and about 31 nucleotides in length.

The terms “shRNA” as used herein refers to a nucleic acid molecule comprising at least two complementary portions hybridized or capable of specifically hybridizing to form a duplex structure sufficiently long to mediate RNAi (typically between about 15 to about 29 nucleotides in length), and at least one single-stranded portion, typically between approximately 1 and about 10 nucleotides in length that forms a loop connecting the ends of the two sequences that form the duplex.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs.

The terms “sgRNA” and “guideRNA” are interchangeable and refer to a specific RNA sequence that recognizes the target DNA region of interest and directs the endonuclease there for editing. The gRNA is usually made up of two parts: crispr RNA (crRNA), a 17-20 nucleotide sequence complementary to the target DNA, and a tracr RNA, which serves as a binding scaffold for a Cas nuclease.

Preferably, the expression of the lncRNA of the invention is downregulated by using a gene editing system such as, e.g. the CRISPR-based gain/loss-of-function system. Usually, the CRISPR-based gain/loss-of-function system comprises at least one single guide RNA (sgRNA), or crRNA and tracrRNA, and a structure-guided endonuclease such as an RNA-guided endonuclease.

Any suitable naturally occurring, or engineered, RNA-guided endonuclease can be employed as long as it is effective for binding a target DNA and it may be selected from the non-limiting group comprising Cas9, Cas12, Cpfl, and FEN-1. Preferably, the RNA-guided endonuclease is Cas9.

The CRISPR/Cas9 system has become a remarkably flexible tool for genome manipulation over the years. A unique feature of Cas9 endonuclease is its ability to bind target DNA independently of its ability to cleave target DNA.

Within the context of this disclosure, the Cas9 endonuclease is preferably a modified Cas9 endonuclease such as, e.g. an enzymatically dead Cas9. Specifically, both RuvC- and/or HNH-nuclease domains can be rendered inactive by point mutations (e.g. D10A and H840A in SpCas9), resulting in a nuclease dead Cas9 molecule that cannot cleave target DNA. However, the dead Cas9 molecule retains the ability to bind to target DNA based on the sgRNA targeting sequence, which sgRNA sequence is comprised in CRISPR-based gain/loss-of-function system.

In one aspect, the enzymatically dead Cas9 is tagged with one or more transcriptional repressor (see Andriy Didovyk, Bartlomiej Borek, Lev Tsimring, and Jeff Hasty. Curr Opin Biotechnol. 2016 August; 40: 177-184 which is incorporated herein by reference).

In another aspect, the enzymatically dead Cas9 is tagged with one or more epitope that is/are recognized by one or more antibody-activator/repressor effector. This enzymatically tagged dead Cas9 can then target the regulatory sequence resulting in robust transcription repression downstream target gene encoding the lncRNA of the invention.

As used herein, the term “target DNA” refers to the gene encoding the lncRNA of the invention as disclosed above or to a regulatory sequence that controls the transcription of the gene encoding the lncRNA of the invention.

Designing and selecting a suitable siRNA, a shRNA, a miRNA, a tRNA, a piRNA, a hnRNA, a snRNA, a sgRNA used in a CRISPR-based loss- or gain-of-function system, an esiRNA, a single-stranded DNA, and an antisense oligonucleotide is well within the competences of one of ordinary skill in the art using routine experimentation, several commercial and noncommercial web sites available for nucleic acid design as well as the information provided herein (for a review e.g. Glen F. Deleavey, et al., Designing Chemically Modified Oligonucleotides for Targeted Gene Silencing, Chemistry & Biology, Volume 19, Issue 8, 2012, Pages 937-954).

In one aspect, the antisense oligonucleotide selectively targeting said lncRNA, fragment, isoform or variant thereof, is a modified antisense oligonucleotide. Preferably, the modified antisense oligonucleotide is a GapmeR or a GapmeR with fixed chemical modification architectures. More preferably, the GapmeR and/or GapmeR with fixed chemical modification selectively targets an exon of the lncRNA, fragment, isoform or variant thereof.

In an aspect, the antisense oligonucleotide selectively targeting said gene encoding the lncRNA of the invention, as disclosed above, or a regulatory sequence that controls the transcription of the gene encoding the lncRNA of the invention is a modified antisense oligonucleotide. Preferably, the modified antisense oligonucleotide is a GapmeR or a GapmeR with fixed chemical modification architectures. More preferably, the GapmeR and/or GapmeR with fixed chemical modification selectively targets an exon or an intron in the gene encoding the lncRNA of the invention as disclosed above or in the regulatory sequence that controls the transcription of the gene encoding the lncRNA of the invention.

Exons are, for example, located between position 121430907-121431522 and 121443988-121444826 on human chromosome 5.

A GapmeR with fixed chemical modification architectures is usually selected from the group comprising i) a gapmer with five 2′-O-methoxyethyl (MOE) modifications in each flank, and a central gap of 10 unmodified dans (e.g. 5-10-5 MOE design), and ii) a gapmer employing three or four locked nucleic acid (LNA) modifications in each flank (e.g. 3-10-3 or 4-8-4 LNA designs), or a combination of one or more thereof (for a review e.g. Natalia Papargyri, et al., Chemical Diversity of Locked Nucleic Acid-Modified Antisense Oligonucleotides Allows Optimization of Pharmaceutical Properties, Molecular Therapy—Nucleic Acids, Volume 19, pages 706-717, 2020).

Non-limiting examples of GapmeRs include the human FIXER LNA GapmeRs (5′-AATCTCAATTCCCGCA, SEQ ID No. 12, 5′-TTACAGTAATCCAGTT, SEQ ID No. 13, 5′-TTAGGAAGGCATGCAA, SEQ ID No. 14) and mouse FIXER LNA GapmeR (AGGCGATTAAGTATGA, SEQ ID No. 15).

Scrambled GapmeRs are used as control. An example of a scrambled/control GapmeR includes the GapmeR as set forth in SEQ ID No. 16 (AACACGTCTATACGC)

As used herein, a “chemical agent” is a compound that produces change by virtue of its chemical composition and its effects on living tissues and organisms. The chemical agent may be a small molecule inhibitor (SMI) and is preferably a non-peptidyl molecule modulating the expression and/or activity of the lncRNA of the invention, fragment, isoform or variant thereof.

The chemical agents of the invention can be tested using a number of techniques known to those of skill in the art.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

In case the agent of the invention is a peptide, then it will preferably be conjugated to an agent that increases the accumulation of the peptide, e.g. in a cardiac cell.

As used herein, an “antibody” is a protein molecule that reacts with a specific antigenic determinant or epitope and belongs to one or five distinct classes based on structural properties: IgA, IgD, IgE, IgG and IgM. The antibody may be a polyclonal (e.g. a polyclonal serum) or a monoclonal antibody, including but not limited to fully assembled antibody, single chain antibody, antibody fragment, and chimeric antibody, humanized antibody as long as these molecules are still biologically active and still bind to at least one peptide or protein of the invention. Preferably the antibody is a monoclonal antibody. Preferably also the monoclonal antibody will be selected from the group comprising the IgG1, IgG2, IgG2a, IgG2b, IgG3 and IgG4 or a combination thereof. Most preferably, the monoclonal antibody is selected from the group comprising the IgG1, IgG2, IgG2a, and IgG2b, or a combination thereof.

An “antigen binding fragment” comprises a portion of a full-length antibody. Examples of antigen binding fragments include Fab, Fab′, F(ab′)2, and Fv fragments; diabodies; linear antibodies; single-chain antibody molecules; and multispecific antibodies formed from antibody fragments.

Usually, the antibody or an antigen-binding fragment i) inhibits and/or impairs the binding of the lncRNA of the invention, fragment, isoform or variant thereof to a target genomic DNA (e.g. by specifically and selectively impairing regulatory role for FIXER on the expression of Lox, Col1a1, Col1a3, Postn or Fn1), and/or the binding of the lncRNA of the invention, fragment, isoform or variant thereof to its interacting protein(s) or other molecule(s).

The present invention also contemplates a gene delivery vector, preferably in the form of a plasmid (circular or linear plasmid), or a vector, that comprises one or more nucleic acid(s) of the invention. Preferably, the one or more nucleic acid is an acid nucleic encoding an agent modulating the expression and/or activity of at least one lncRNA, a fragment thereof, an isoform thereof and a variant sharing at least 80% nucleotide sequence identity thereto, encoded by a DNA sequence comprising the sequence of SEQ ID No. 1, a fragment thereof, and a variant sharing at least 80% nucleotide sequence identity thereto.

Preferably, said one or more nucleic acid of the invention encodes a siRNA, a shRNA, a miRNA, a transfer RNA (tRNA), a piRNA, a heterogeneous nuclear RNA (hnRNA), an snRNA, an sgRNA used in a CRISPR-based loss- or gain-of-function system, an esiRNA, a single-stranded DNA, and an antisense oligonucleotide, or a fragment of one thereof or a combination of one or more thereof.

As used herein, a “vector” or a “gene delivery vector” is capable of transferring nucleic acid sequences to target cells (e.g., viral vectors, non-viral vectors, particulate carriers, and liposomes).

Suitable vectors include derivatives of SV40 and known bacterial plasmids, e. g., E. coli plasmids col El, pCR1, pBR322, pMB9 and their derivatives, plasmids such as RP4; phage DNAs, e. g., the numerous derivatives of phage X, e. g., NM989, and other phage DNA, e. g., M1 3 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like (for a review, Sung, Y., Kim, S. Recent advances in the development of gene delivery systems. Biomater Res 23, 8 (2019)).

Various viral vectors are used for delivering nucleic acids to cells in vitro or in vivo. Non-limiting examples are vectors based on Herpes Viruses, Pox-viruses, Adeno-associated virus, Lentivirus, and others. In principle, all of them are suited to deliver the expression cassette comprising an expressible nucleic acid molecule that codes for an agent modulating the expression and/or activity of a lncRNA having a cDNA sequence selected from the group comprising the sequence of SEQ ID No. 1, a fragment thereof, an isoform thereof and a variant of the invention. In a preferred aspect, said viral vector is an adenoviral vector, preferably a replication competent adenovirus.

The present invention also contemplates a host cell, whether eucaryotic or procaryotic, comprising i) a plasmid or vector of the invention, ii) one or more nucleic acid(s) encoding the siRNA, miRNA, piRNA, hnRNA, snRNA, sg RNA, CRISPR-based loss-of-function system, esiRNA, shRNA, and antisense oligonucleotide, or a combination of one or more thereof, of the invention, or iii) a siRNA, miRNA, piRNA, hnRNA, snRNA, sg RNA, CRISPR-based loss-of-function system, esiRNA, shRNA, and antisense oligonucleotide, or a combination of one or more thereof, of the invention.

The gene delivery vector (e.g. plasmid or vector) comprising i) one or more nucleic acid(s) encoding, comprising, or consisting of, a siRNA, miRNA, piRNA, hnRNA, snRNA, sg RNA, CRISPR-based loss-of-function system, esiRNA, shRNA, and antisense oligonucleotide, or combination of one or more thereof, of the invention, ii) or the one or more nucleic acid(s) of the invention, can be introduced to host cell via one or more methods known in the art. These one or more methods include, without limitation, microinjection, electroporation, calcium phosphate-mediated transfection, cationic transfection, liposome transfection, dendrimer transfection, heat shock transfection, nucleofection transfection, magnetofection, lipofection, optical transfection, proprietary agent-enhanced uptake of nucleic acids, and delivery via liposomes, immunoliposomes, virosomes, or artificial virions.

It will be appreciated that in the present method the modification following the introduction of the gene delivery vector (plasmid or vector), or the one or more nucleic acid(s) of the invention, to the host cell may occur ex vivo or in vitro, for instance in a cell culture and in some instances not in vivo. In other aspects, it may occur in vivo.

The present invention also contemplates compositions as well as pharmaceutical compositions.

In an aspect of the invention, the composition comprises at least one i) agent of the invention, ii) vector of the invention, iii) nucleic acid of the invention, and/or iv) host cell of the invention.

In an aspect of the invention, the pharmaceutical composition comprises a therapeutically effective amount of at least one i) agent of the invention, ii) vector of the invention, iii) nucleic acid of the invention, and/or iv) host cell of the invention, in combination with pharmaceutically acceptable carriers, diluents and/or adjuvants.

The term “therapeutically effective amount” as used herein means an amount of an agent, vector, nucleic acids, compound, and/or host cell high enough to significantly positively modify the symptoms and/or condition to be treated, but low enough to avoid serious side effects (at a reasonable risk/benefit ratio), within the scope of sound medical judgment.

The therapeutically effective amount of an agent, vector, nucleic acids, compound, and/or host cell as described herein is selected in accordance with a variety of factors including type, species, age, weight, sex and medical condition of the patient; the severity of the condition to be treated; the route of administration; the renal and hepatic function of the patient. A physician of ordinary skill in the art can readily determine and prescribe the effective amount of the drug required to prevent, counter or arrest the progress of the disease, i.e. cardiac disease.

“Pharmaceutically acceptable carrier or diluent” means a carrier or diluent that is useful in preparing a pharmaceutical composition that is generally safe, non-toxic, and desirable, and includes carriers or diluents that are acceptable for human pharmaceutical use.

Such pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Pharmaceutically acceptable excipients include starch, glucose, lactose, sucrose, sodium stearate, glycerol monostearate, talc, sodium chloride, dried skim milk, glycerol, propylene glycol, water, ethanol and the like.

The pharmaceutical compositions may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include macrocrystalline cellulose, carboxymethyf cellulose sodium, polysorbate 80, phenyletbyl alcohol, chiorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

The pharmaceutical composition of the invention can further comprise at least one additional therapeutic agent or therapy useful to treat a cardiac disease or another disease.

Also contemplated in the present invention is at least one lncRNA, fragment, isoform or variant thereof encoded by a DNA sequence comprising the sequence of SEQ ID No. 1, a fragment thereof, and a variant sharing at least 80% nucleotide sequence identity thereto.

Further contemplated is a method of treating and/or preventing a cardiac pathology in a subject in need thereof, comprising administering to said subject a therapeutically effective amount of an agent modulating the expression and/or activity i) of at least one lncRNA, a fragment thereof, an isoform thereof and a variant sharing at least 80% nucleotide sequence identity thereto, encoded by a DNA sequence comprising the sequence of SEQ ID No. 1, a fragment thereof, and a variant sharing at least 80% nucleotide sequence identity thereto, or
ii) of a DNA sequence comprising the sequence of SEQ ID No. 1, a fragment thereof, and a variant sharing at least 80% nucleotide sequence identity thereto, wherein said DNA sequence encodes at least one lncRNA, a fragment thereof, an isoform thereof and a variant sharing at least 80% nucleotide sequence identity thereto,
or iii) of a pharmaceutical composition comprising a therapeutically effective amount of an agent of the invention, a vector of the invention, or a host cell of the invention, in combination with pharmaceutically acceptable carriers, diluents and/or adjuvants.

The cardiac pathology is as described herein.

“Administering”, as it applies in the present invention, refers to contact of an effective amount of an agent (e.g. modulator) of the invention, to the subject.

The dose or amount to be administered will vary depending upon the age, weight, and general condition of the subject as well as the severity of the condition being treated, the judgment of the health care professional, and conjugate being administered. Therapeutically effective amounts can be determined by those skilled in the art and will be adjusted to the particular requirements of each particular case. Generally, a therapeutically effective amount will range from about 0.50 mg to 5 grams daily, more preferably from about 5 mg to 2 grams daily, even more preferably from about 7 mg to 1.5 grams daily.

In certain aspects, multiple therapeutically effective doses of each of at least one agent of the invention will be administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved. The agents can be administered by any acceptable route of administration as noted herein below.

An agent of the invention (e.g. modulator) can be administered prior to, concurrent with, or subsequent to at least one additional therapeutic agent. If provided at the same time as the additional therapeutic agent, the agent modulating the expression and/or activity i) of the lncRNA of the invention or ii) the gene encoding said lncRNA can be provided in the same or in a different composition. Thus, the agents can be presented to the individual by way of concurrent therapy. By “concurrent therapy” is intended administration to a human subject such that the therapeutic effect of the combination of the substances is caused in the subject undergoing therapy. For example, concurrent therapy may be achieved by administering at least one therapeutically effective dose of a pharmaceutical composition comprising a lncRNA modulator and at least one therapeutically effective dose of a pharmaceutical composition comprising at least one additional therapeutic agent according to a particular dosing regimen. Administration of the separate pharmaceutical compositions can be at the same time (i.e., simultaneously) or at different times (i.e., sequentially, in either order, on the same day, or on different days), so long as the therapeutic effect of the combination of these substances is caused in the subject undergoing therapy.

In other aspects of the invention, the pharmaceutical composition of the invention is a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition. The pharmaceutical compositions of the invention may be administered using the same or different routes of administration in accordance with any medically acceptable method known in the art. Suitable routes of administration include parenteral administration, such as subcutaneous (SC), intraperitoneal (IP), intramuscular (IM), intravenous (IV), or infusion, oral and pulmonary, nasal, topical, transdermal, and suppositories. Where the composition is administered via pulmonary delivery, the therapeutically effective dose is adjusted such that the soluble level of the agent of the invention (i.e. lncRNA modulator) is equivalent to that obtained with a therapeutically effective dose that is administered parenterally, for example SC, IP, IM, or IV. In some aspects of the invention, the pharmaceutical composition comprising the lncRNA modulator is administered by IM or SC injection, particularly by IM or SC injection locally to the region where the therapeutic agent or agents used in the cardiac therapy protocol are administered.

Factors influencing the respective amount of the various compositions to be administered include, but are not limited to, the mode of administration, the frequency of administration (i.e., daily, or intermittent administration, such as twice- or thrice-weekly), the particular disease undergoing therapy, the severity of the disease, the history of the disease, whether the individual is undergoing concurrent therapy with another therapeutic agent, and the age, height, weight, health, and physical condition of the individual undergoing therapy. Generally, a higher dosage of this agent is preferred with increasing weight of the subject undergoing therapy.

Another aspect of the invention relates to a kit comprising i) a composition comprising the lncRNA modulator wherein the modulator or agent of the invention is selected from the group comprising a chemical agent, a RNA mimic, an antibody, an engineered protease, and enzymatically active RNA or ii) a pharmaceutical composition comprising an effective amount of the lncRNA modulator wherein the modulator or agent of the invention is selected from the group comprising a chemical agent, a RNA mimic, an antibody, an engineered protease, and enzymatically active RNA, optionally in combination with pharmaceutically acceptable carriers, diluents and/or adjuvants.

In yet another aspect, the invention provides kits for use in diagnosing a cardiac pathology or a cardiac tissue undergoing regeneration or a stem cell undergoing cardiac differentiation or a cardiac tissue undergoing surgical and/or pharmacological therapies, wherein the kits can be used to detect the lncRNA of the present invention. For example, the kits can be used to detect any one or more of the lncRNA, fragment, isoform or variant thereofs described herein, which are differentially expressed in samples of a patient with a cardiac pathology or a cardiac tissue undergoing regeneration or a stem cell undergoing cardiac differentiation or a cardiac tissue undergoing surgical and/or pharmacological therapies. The kit may include i) one or more agents for detection of the lncRNA, fragment, isoform or variant thereof described herein, ii) a container for holding a biological sample isolated from a human subject; and iii) printed instructions for reacting agents with the biological sample or a portion of the biological sample to detect the presence or amount of at least one lncRNA biomarker in the biological sample. The agents may be packaged in separate containers. The kit may further comprise one or more control reference samples and reagents for performing an immunoassay, a Northern blot, PCR, microarray analysis, or SAGE, DNA/RNA-sequencing,

In certain aspects, the kit contains at least one probe that selectively hybridizes to the lncRNA, fragment, isoform or variant thereof described herein, or at least one antibody that selectively binds to a lncRNA, fragment, isoform or variant thereof, or at least one set of PCR primers for amplifying a lncRNA, fragment, isoform or variant thereof selected from the group comprising selected from the group comprising a sequence of SEQ ID No. 2, SEQ ID No. 3, SEQ ID No. 4, SEQ ID No. 5, SEQ ID No. 6, SEQ ID No. 7, SEQ ID No. 8, and SEQ ID No. 9, or a combination of one or more thereof.

In one aspect, the kit comprises at least one agent for measuring the level of a lncRNA, fragment, isoform or variant thereof.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing a cardiac pathology or monitoring stem cell therapy or regenerative medical treatments.

The kits of the invention have number of applications. For example, the kits can be used for diagnosing a cardiac pathology or monitoring and/or evaluating the efficacy of a treatment for a cardiac pathology, stem cell therapy, or regenerative cardiac medicine. In a further example, the kits can be used to identify compounds that modulate expression of one or more of the lncRNA, fragment, isoform or variant thereofs in in vitro or in vivo animal models to determine the effects of treatment.

The present invention also contemplates the use of the compositions or pharmaceutical compositions of the invention in the manufacture of a medicament for the treatment of a cardiac pathology described herein.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); REMINGTON'S PHARMACEUTICAL SCIENCES (Mack Pub. Co., N.J. 1991); Carey and Sundberg Advanced Organic Chemistry 3.sup.rd Ed. (Plenum Press) Vols A and B (1992).

The present disclosure is therefore to be considered as in all aspects illustrated and not restrictive, the scope of the invention being indicated by the appended Claims, and all changes which come within the meaning and range of equivalency are intended to be embraced therein.

### EXAMPLES

### Material and Methods

### Animal Experiments

Mouse experiments have been approved by the Government Veterinary Office (Lausanne, Switzerland) and conducted according to the University of Lausanne institutional guidelines and the Swiss laws for animal protection.

### Mouse Model of Myocardial Infarction (MI)

MI was performed by placing a permanent ligation on the left anterior descending (LAD) coronary artery. Male 10-12 week-old C57BL/6 mice (Charles River) were housed in a 12-hour light/12-hour dark cycle at a temperature of 23° C. with 40 to 60% humidity, and fed ad libitum. Mice were anesthetized with ketamine/xylazine/acepromazine (65/15/2 mg/kg; i.p.) and placed on artificial ventilation. Following thoracotomy, the pericardium was opened and a 7.0 silk ligature was tied around the LAD artery. Successful occlusion was confirmed by rapid blanching of the left ventricle. Buprenorphine was administered (0.1 mg/kg; s.c.). Mice were gradually weaned from the respirator until spontaneous respiration resumed. Sham-operated mice underwent to the same procedure involving thoracotomy without LAD ligation.

Non-Myocyte Cells Isolation from Sham-Operated and Infarcted Mouse Hearts

Mice were injected with 100 U Liquerin, 15 min before sacrifice to avoid coagulation. Beating hearts were removed and placed in ice cold PBS. The aorta was cannulated. Hanks' Balanced Salt Solution (HBSS) without Ca2⁺ and Mg2⁺ was infused to flush out the blood from the heart. Next, a solution of digestion enzymes (Pierce Primary Cardiomyocyte Enzyme, Thermo scientific, cat. 88281) was pumped into the heart. This step was followed by 10-min digestion period at 37° C. The whole process was repeated 3-4 times. Then, the atria and large vessels were eliminated. The ventricles were minced and resuspended in DMEM. The tissue was pipetted up and down 15-20 times to reach a single cell suspension. The suspension was passed through a 300-μm strainer. The cells were gently centrifuged (200 rpm, 5 min) to remove cardiomyocytes. The supernatant was harvested and passed through a 40-μm strainer. Non-myocyte cells were collected by centrifugation at 800 rpm for 5 min. The final cell pellets were resuspended in DMEM 10% FBS.

### Fluorescence-Activated Cell Sorting (FACS)

FACS was used to remove CD45⁺ leukocytes and CD31⁺ endothelial cells from Calcein⁺ metabolically active and DAPI⁻ viable non-myocyte cell suspension. Briefly, non-myocyte cells were incubated with anti-CD45 (Biolegends, cat. 103112) and anti-CD31 antibodies (Biolegends, cat. 102408) at 4° C. for 30 min, and washed twice with PBS 1% FBS. Cells were next stained with Calcein (BD Biosciences, cat. 564061) at 37° C. for 10 min. and washed twice with PBS 1% FBS. DAPI was added to cell suspensions before running samples into the flow cytometer. Viable CD45-CD31—non-myocyte cells were analyzed by single-cell RNA-Sequencing.

Single-Cell RNA-Sequencing (scRNA-Seq)

For each scRNA-Seq sample, cells were pooled from four mouse hearts. Cells were counted using a hemocytometer. Trypan blue was used for checking viability (<80%). Cells were loaded in a Chromium Next GEM Chip (10× Genomics). Sequencing libraries were prepared according to instructions. Barcoded Gel Beads are mixed with cells and partitioning oil to form tens of thousands of single cell emulsion. After barcoding, all fragments from the same cell share a common 10× Barcode. Barcoded fragments for thousands of cells are pooled for downstream reactions to create short-read sequencer compatible libraries. Libraries were quantified and assessed on a Fragment Analyzer (Agilent Technologies). Sequencing was performed on an Illumina HiSeq 4000 using HiSeq 3000/4000 SBS Kit reagents. Sequencing data were demultiplexed using the bcl2fastq2 Conversion Software (v. 2.20, Illumina) and primary data analysis performed with the Cell Ranger Gene Expression pipeline (version 3.0.2, 10× Genomics).

### Single-Cell RNA-Sequencing Data Analysis

Cell Ranger 1.3 (10× Genomics) was use to generate FASTQ files from raw scRNA-Seq data. FASTQ files were aligned to the mouse mm10/GRCm38 reference genome. Gene expression matrices were created. Downstream bioinformatic analysis was performed in R using the Seurat v.3 (Stuart et al., 2019). Dimensional reduction of the data was performed by RunUMAP function implemented in the Seurat package using the Uniform Manifold Approximation and Projection (UMAP) algorithm.

### Tissue Collection

Mouse hearts were harvested at 3, 6, 9, 12 and 15 days post-infraction. The ventricles were rinsed with PBS, snap frozen in liquid nitrogen and stored at −80° C. until used. Human cardiac fibroblasts were obtained from human adult atria from patients undergoing surgery.

Isolation of Cardiac Fibroblast from Neonatal Mouse Hearts

Neonatal C57B6 mice were sacrificed within 24-48 h after birth, and beating hearts were collected. The atria and large vessels were eliminated. The ventricles were minced into 1-3 mm² pieces and washed twice in ice-cold HBSS. Minced tissues were placed in a 1.5-ml tubes (5-6 hearts per tube) containing a solution with digestion enzymes (Pierce Primary Cardiomyocyte Enzyme, Thermo scientific, cat. 88281), and incubated at 37° C. with shaking for 30 min. Next, the enzymatic solution was removed and tissues were washed with ice-cold HBSS. Complete medium was added to each tube and the solution was pipetted up and down 10-20 times to reach a single cell suspension. Cells were then plated in 10-cm dishes for 45 min at 37° C. (pre-plating 1). Non-myocyte cells adhere to the plastic whereas the supernatant is enriched in cardiomyocytes. After 45 min, the supernatant was transferred to a new 10-cm dish and the process was repeated (pre-plating 2). Fresh complete medium was added to each pre-plating dish to culture non-myocytes cells.

### Gapmer Transfection in Neonatal Mouse Cardiac Fibroblast Cells

Neonatal mouse non-myocyte cells or human cardiac fibroblasts were used after 1-3 passages. Cells were transfected with LNA lncRNA Gapmers described herein (25 nM, Qiagen) using Lipofectamine LTX (Invitrogen, cat. A12621). After 48 hours, medium was changed, and cells were collected for analysis.

RNA Isolation, cDNA Synthesis and Quantitative RT-PCR Analysis

Total RNA was isolated from mouse or human tissues or cultured cells using miRNeasy kit (Qiagen, cat. 217084). Complementary (c)DNA was synthetized using PrimeScript™ RT reagent kit (Takara, RR037A). Quantitative RT-PCR (qRT-PCR) was performed using Tagman™ Universal PCR Master Mix (Applied Biosystems, cat.4324020) or Power SYBR™ Green PCR master mix (Applied Biosystems, cat.4367659), with gene specific probes or primers. Examples of pèrimers and probes are listed in Table 1 and Table 2. Raw values were normalized to Gapdh. Mean fold differences were calculated by the comparative Ct method (2^{-ΔΔCt}).

### Transcriptome Analysis Using RNA Sequencing

Total RNA was extracted from sham-operated and infarcted mouse hearts. RNA quality was assessed on a Fragment Analyzer (Agilent Technologies). RNA-Seq libraries were prepared using 500 ng of total RNA with the Illumina TruSeq Stranded mRNA reagents, using a unique dual indexing strategy. Libraries were quantified by a fluorimetric method (QubIT, Life Technologies). Quality was assessed on a Fragment Analyzer (Agilent Technologies). Cluster generation was performed with 2 nM of an equimolar pool from the resulting libraries using the Illumina HiSeq 3000/4000 SR Cluster Kit reagents and were sequenced on the Illumina HiSeq 4000 using HiSeq 3000/4000 SBS Kit reagents for 150 cycles (single-end method). Sequencing data were demultiplexed using the bcl2fastq2 Conversion Software (v. 2.20, Illumina).

### RNA-Seq Data Analysis

The RNA-Seq data was aligned ton the mouse mm10/GRCm38 reference genome and analyzed using Qlucore Omics Explorer base module v. 3.7 (Qlucore, Sweden). Trimmed mean of M values (TMM) normalization was used to assess the level of gene expression. The dataset was analyzed by two-way ANOVA. Genes with significant difference in their expression at p-value<0.05 and fold differences ≥1.5 were selected. Principal Component Analysis (PCA) was used to visualize the data in a three-dimensional space, after filtering out variables with low overall variance to reduce the impact of noise, and centering and scaling the remaining variables to zero mean and unit variance. Unsupervised hierarchical clustering of significant upregulated or downregulated genes were applied using the standard Euclidean's method and the heat maps were generated according to the standard normal distribution of the values. Pathway enrichment analysis was performed using Enrichr (Chen et al., 2013) and visualized by circus plot using Circa software (https://gumroad.com/l/circa).

### Lox Activity Assay

Lox activity was measured using a fluorometric Lysyl Oxidase assay Kit (AAT Bioquest, cat. 15255) according to the manufacturer's instructions.

### GapmeR Administration In Vivo

Mouse Fixer GapmeR and Scrambled GapmeR (Qiagen), as described herein, were diluted in NaCl 0.9%. GapmeRs were administered at a dose of 10 mg/kg, i.p. at 2, 4 and 9 days post injury.

### Histological Evaluation of Cardiac Fibrosis

Mouse hearts were harvested, and atria and large vessels were eliminated. Cardiac tissues were embedded in OCT medium. Cryosections were processed for Masson's trichrome staining (MTS) using standard histological procedures and analyzed with a Zeiss Axioscan Z1 (Carl Zeiss). The percentage of fibrotic tissue was determined by measuring collagen deposition (blue) in Masson's trichrome-stained sections.

### Echocardiographic Analysis

Transthoracic echocardiographies were performed using a 30-MHz probe and a Vevo 770 Ultrasound machine (VisualSonics, Canada). Left ventricular free wall thickness in diastole (LVWT; d) and in systole (LVWT; s), left ventricle diameter in diastole (LVD; d) and in systole (LVD; s), Ejection fraction (EF) and infarct size were determined.

### Statistical Analysis

Statistical analysis was performed using GraphPad Prism® software v8.3.0 (GraphPad software, USA). Data throughout the paper are expressed as mean±SEM. The Shapiro-Wilk test was used to assess the assumption of normality in the datasets. Two-tailed Student's t-test and one-way-ANOVA followed by Tukey's post-test were used as indicated in the legends. Difference between experimental groups was considered significant when p<0.05.

TABLE 2
TaqMan probes

| Species | Gene Name | Reference | Supplier |
|---|---|---|---|
| Mouse | Gapdh | Mm99999915_g1 | Applied biosystem |
| Mouse | Lox | Mm00495386_m1 | Applied biosystem |
| Mouse | Postn | Mm01284919_m1 | Applied biosystem |
| Mouse | Col1a1 | Mm00801666_g1 | Applied biosystem |
| Mouse | Col3a1 | Mm00802331_m1 | Applied biosystem |
| Mouse | Fn1 | Mm00692666_m1 | Applied biosystem |
| Mouse | Nppa | Mm01255747_g1 | Applied biosystem |
| Human | GAPDH | Hs99999905_m1 | Applied biosystem |
| Human | ACTA2 | Hs00909449_m1 | Applied biosystem |
| Human | COL1A1 | Hs00164004_m1 | Applied biosystem |
| Human | COL3A1 | Hs00943809_m1 | Applied biosystem |
| Human | LOX | Hs00942483_m1 | Applied biosystem |
| Human | POSTN | Hs01566750_m1 | Applied biosystem |

### Results

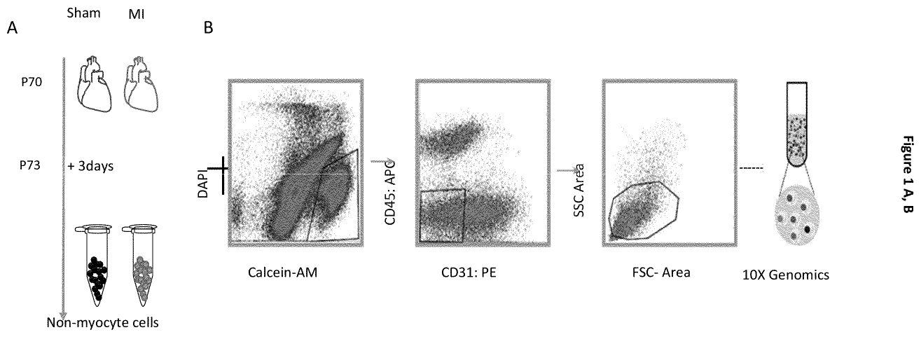

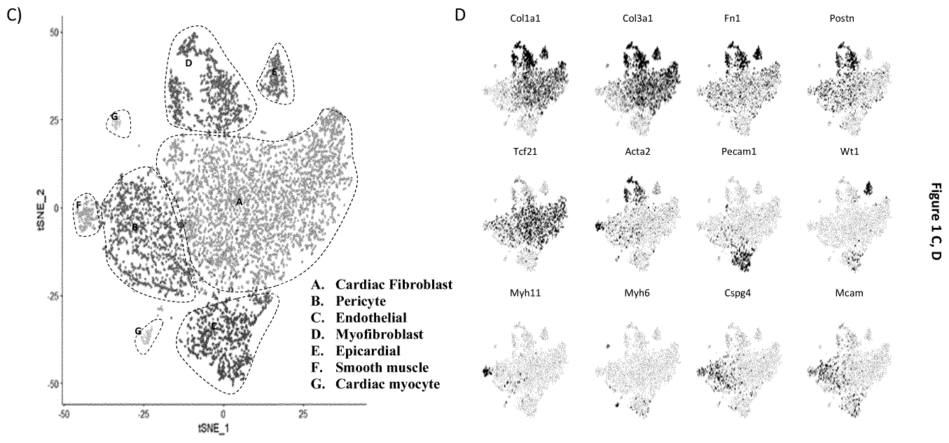

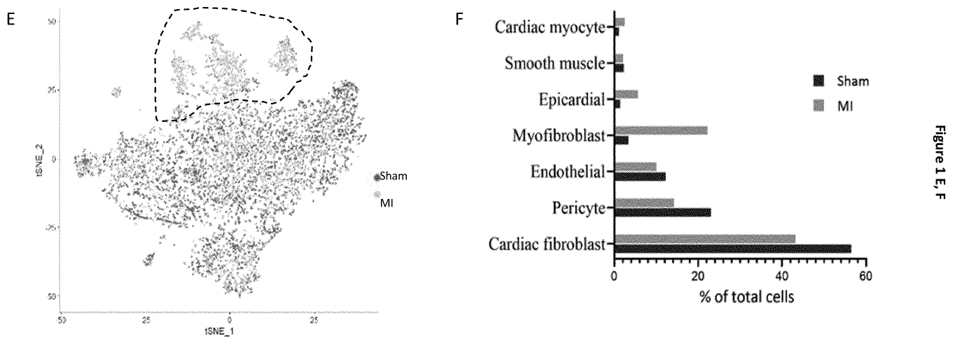

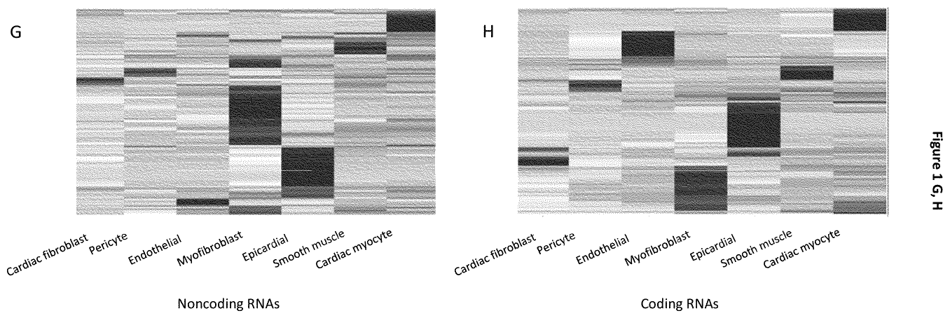

Single-Cell RNA-Sequencing Analysis of Non-Myocyte Cells Isolated from Sham-Operated and Infarcted Mouse Hearts (FIG. 1)

In order to gain insights into the transcriptomic regulation of cardiac fibroblasts (CFs) after myocardial infarction (MI), we performed a single-cell RNA sequencing (scRNA-Seq) analysis of non-myocyte cells isolated from sham-operated and infarcted mouse hearts.

The non-myocyte fraction were isolated from adult mouse hearts using a Langendorff approach three days after MI (FIG. 1A). Cardiomyocytes were removed by sedimentation using gentle centrifugation, and metabolically active (Calcein⁺) and viable (DAPI⁻) non-myocyte cells were separated from leukocytes (CD45⁺) and endothelial cells (CD31⁺) by Fluorescence-activated cell sorting (FACS). Then, sorted cells were subjected to a single-cell analysis using a 10× Genomics platform (FIG. 1B).

An average number of 200,000 reads per cell allowed identifying a total of 73,828 transcripts (coding and noncoding RNAs) in 8,413 cells from either sham-operated or infarcted hearts. We created t-SNE maps to visualize data in low-dimensional space and group cells according to their closest neighbors using only long noncoding (lnc)RNA expression. Importantly, we were able to identify relevant cardiac cell types (clusters) based on lncRNA expression, validating thereby this novel approach (FIG. 1C). Cells belonging to these clusters were identified as cardiac fibroblasts, myofibroblasts, epicardial cells, pericytes, endothelial cells, smooth muscle cells and cardiac myocytes (FIG. 1D). Importantly, some clusters appeared to be only present in the infarcted heart (FIG. 1E; clusters surrounded by a dashed line), and represent therefore prime targets for therapeutic interventions. Percentages of cells per cluster in each sample are shown in FIG. 1F. As expected, more than 50% of cells were cardiac fibroblast. Then, RNA profiling was performed for each cluster, highlighting the enrichment of specific coding and non-coding transcripts in each individual cluster (FIGS. 1G and H; dark blocks indicated blocks of highly expressed transcripts in clusters).

Altogether, the results depicted in FIG. 1 demonstrate that lncRNA expression profiles can be used to identify relevant cardiac cell types, providing thereby a novel approach for identifying candidate lncRNAs specifically expressed in cardiac subpopulations associated with disease. In particular, lncRNAs enriched in myofibroblasts represent prime targets for modulating the development of fibrosis in the heart after MI.

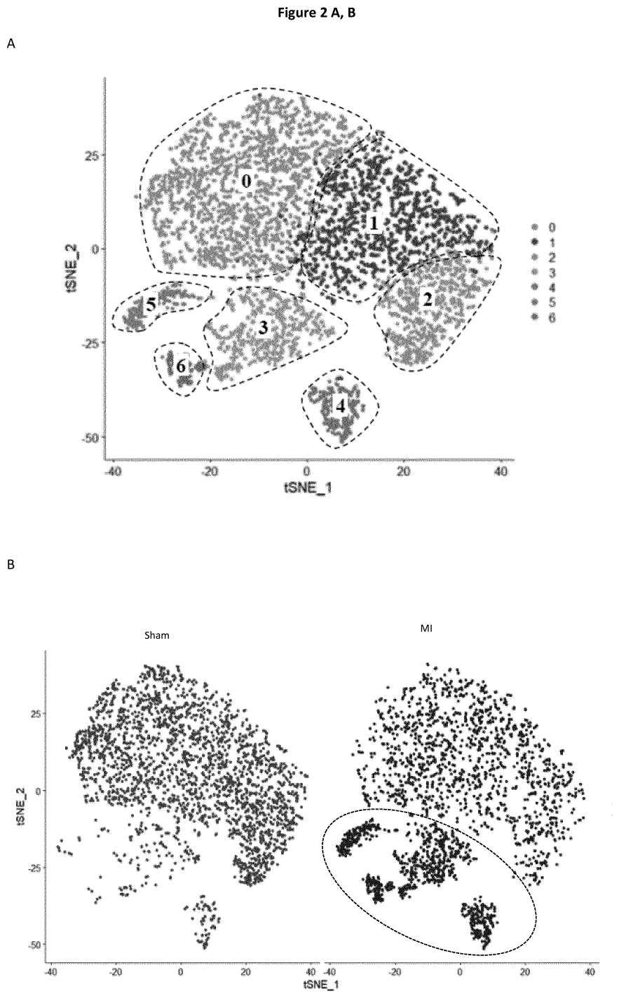

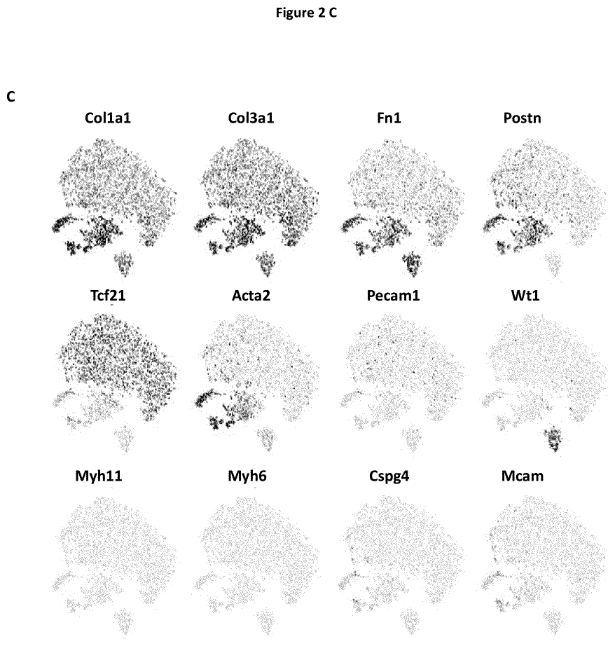

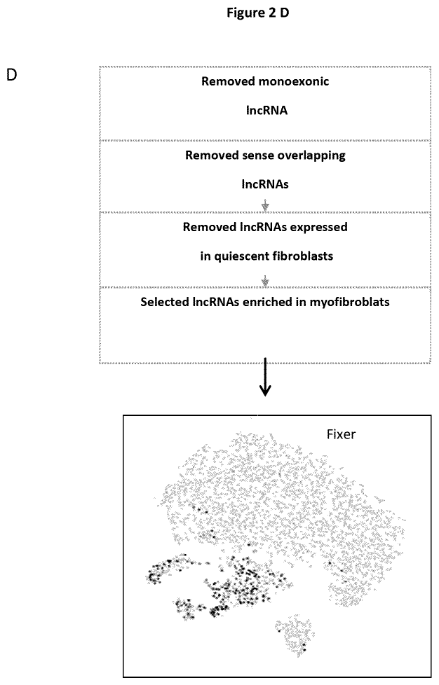

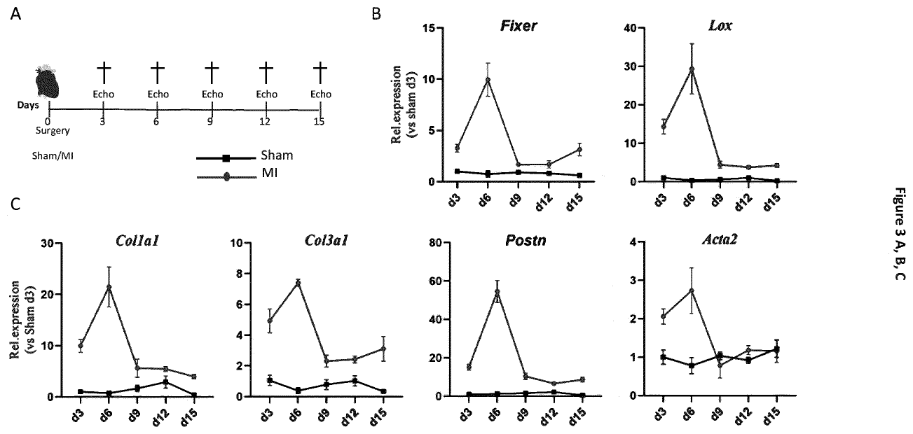

Selection of a New lncRNAs Expressed in Myofibroblasts after MI (FIG. 2)

Since cardiac fibroblasts are critical regulators of the response to myocardial infarction in the adult heart, we focused our analysis on this particular cell type. Of note, scRNA-Seq analysis is the method of choice for evaluating cellular heterogeneity in seemingly homogenous cell populations. Thus, we refined our analysis to probe specifically heterogeneity in cardiac fibroblasts and myofibroblasts. Re-clustering of cardiac fibroblasts revealed transcriptome differences in noncoding RNAs between injured (MI) and control (sham) hearts. We were able to identify seven different subclusters (Clusters 0-6; FIG. 2A), of which four were only present in the infarcted heart (FIG. 2B; clusters surrounded by a dashed line). Expression of cell-specific markers allowed identifying the different cell types, namely quiescent cardiac fibroblasts (Cluster 0, 1 and 2), myofibroblasts (Cluster 3, 5 and 6) and epicardial cells (Cluster 4) (FIG. 2C). Importantly, myofibroblasts are associated to subpopulations present in the infarcted heart. We applied a selection pipeline to bioinformatically identified relevant lncRNAs expressed in myofibroblasts after MI (FIG. 2D). In the end, FIXER, a novel enhancer-associated lncRNA, was selected for functional analysis. FIXER stands for Fibroblast-enriched Lox-locus enhancer-RNA. Indeed, FIXER is located adjacent to the Lox gene encoding a lysyl oxidase implicated in collagen crosslinking, a process regulating stabilization of the fibrotic matrix during the response of the heart to stress.

In summary, using scRNA-Seq in cardiac non-myocyte cells and a stringent selection procedure, we discovered a novel CF-enriched lncRNA, highly upregulated in the heart after MI. The expression of this lncRNA in myofibroblasts suggested that it could represent a novel therapeutic target to reduce fibrosis in the damaged heart.

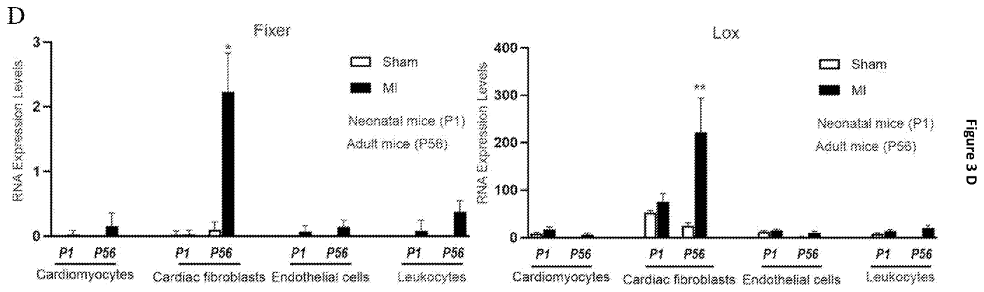

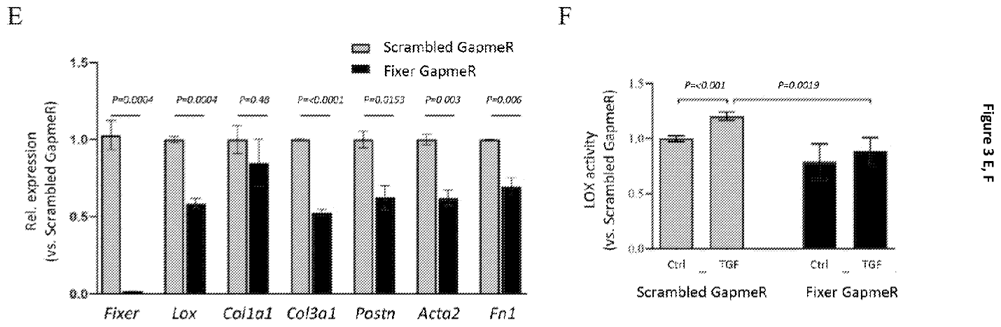

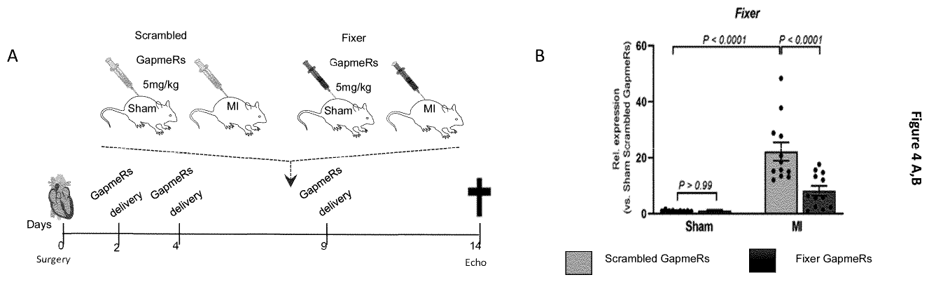

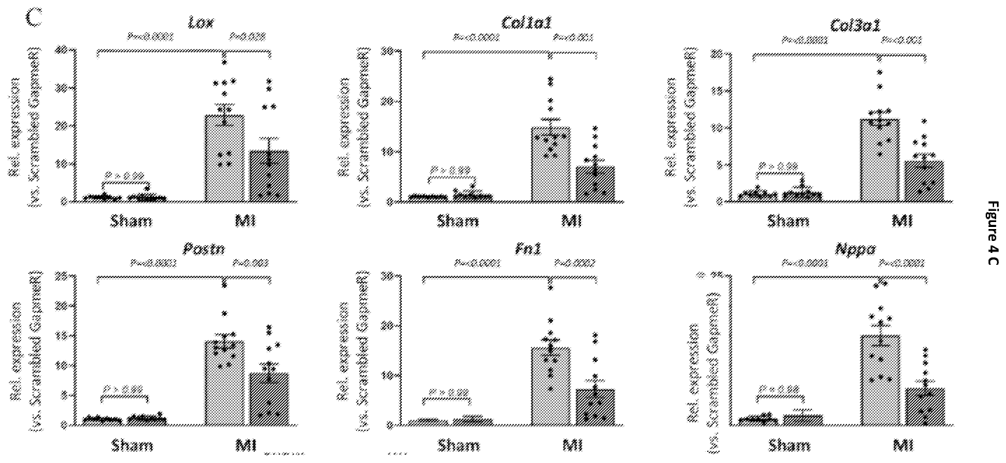

### Time Course of FIXER Expression in the Heart (FIG. 3)

We next determined the time course of FIXER expression in the heart in a mouse model of myocardial infarction (FIG. 3A). RNA was isolated from the heart at different times after MI. The expression of a series of fibrosis marker genes as well as the expression of FIXER and Lox were measured by RT-qPCR. Both FIXER and Lox were induced after MI, with maximal expression observed six days post injury (FIG. 3B). The expression of the transcript correlated significantly with the expression of the fibrosis marker genes (FIG. 3C), suggesting FIXER was implicated in the fibrotic response after cardiac injury. We have shown that FIXER was highly upregulated after myocardial infarction in adult CFs in the adult heart as compared to the neonatal heart (FIG. 3D). Other major cardiac cells showed no significant FIXER expression. Lox was also found to be CF-enriched.

To characterize the functional role of FIXER, we designed antisense oligonucleotides (GapmeRs) to specifically knock down FIXER expression in isolated neonatal murine CFs (FIG. 3E). Transfection of CFs with FIXER-specific GapmeRs abolished FIXER gene expression. Importantly, silencing of FIXER blunted the expression of fibrosis marker genes, and was also associated with Lox downregulation. This suggests Lox is under control by FIXER via a Cis-regulatory mechanism. We also checked LOX protein activity upon FIXER silencing in fibroblasts cultured in the absence or presence of TGF-β. Interestingly, FIXER depletion led to a significant reduction of LOX crosslinking activity (FIG. 3F).

Altogether, these data demonstrate the regulatory role of FIXER on the fibrotic gene program in cardiac fibroblasts and myofibroblasts.

### Therapeutic Potential of FIXER (FIG. 4)

To functionally evaluate the therapeutic potential of FIXER in vivo, anti-FIXER GapmeRs were administered to mice after MI. FIXER-GapmeRs were injected at 2-, 4- and 9-days post injury, during a clinically relevant therapeutic window. Cardiac dimensions and function were assessed by echocardiography after two weeks (FIG. 4A). FIXER expression was highly upregulated after MI in the control group treated with scrambled GapmeRs. In sharp contrast, FIXER expression was largely decreased in mice receiving anti-FIXER-GapmeRs (FIG. 4B). FIXER downregulation was associated with a significant decreased expression of fibrotic genes including Lox, Col1a1, Col1a3, Postn and Fn1. (FIG. 4C). In addition, expression of Nppa was also reduced upon FIXER silencing, suggesting beneficial effects on heart function. Consistently, FIXER silencing reduced infarcted size and interstitial fibrosis (FIGS. 4D and E), corrected cardiac remodeling (FIG. 4F; IVS: intraventricular septum; LVPW: Left ventricular posterior wall; LVID: Left ventricular internal diameter), and significantly improved cardiac function (FIG. 4G; EF: Ejection fraction).

Overall, these results support FIXER being an important novel therapeutic target for cardiac fibrosis and remodeling in the infarcted heart.

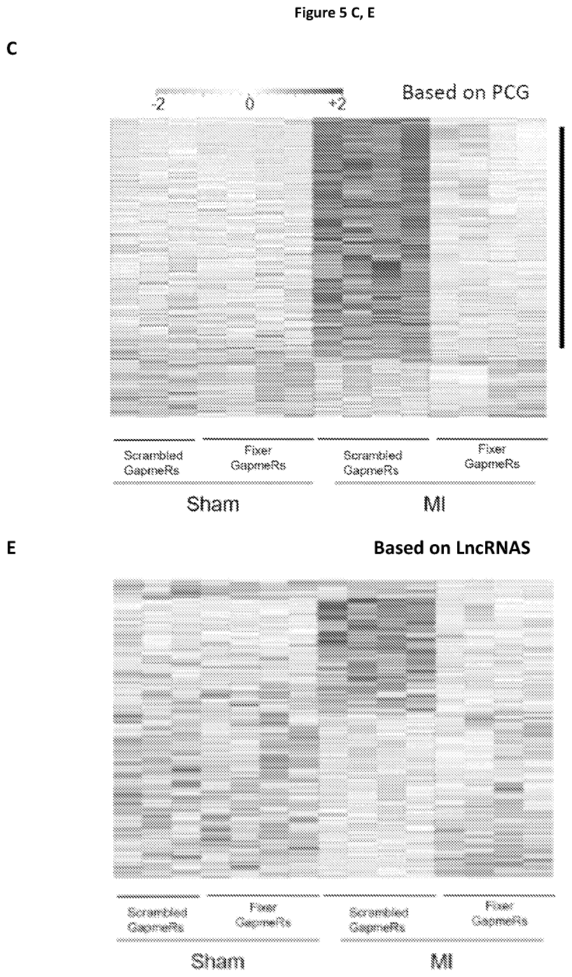

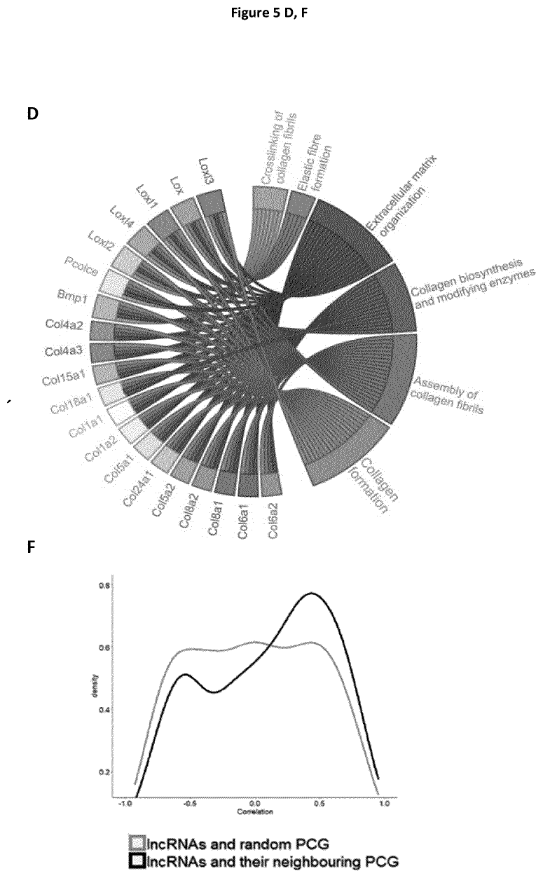

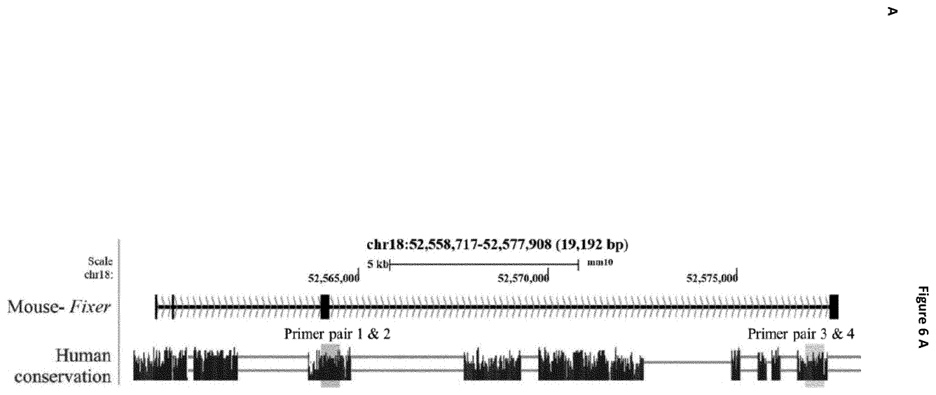

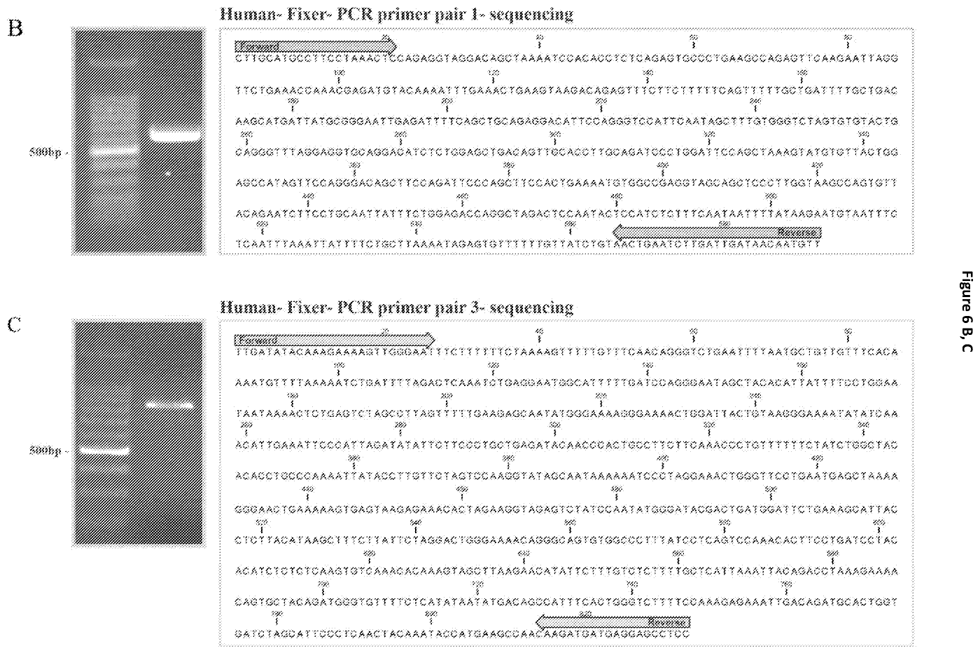

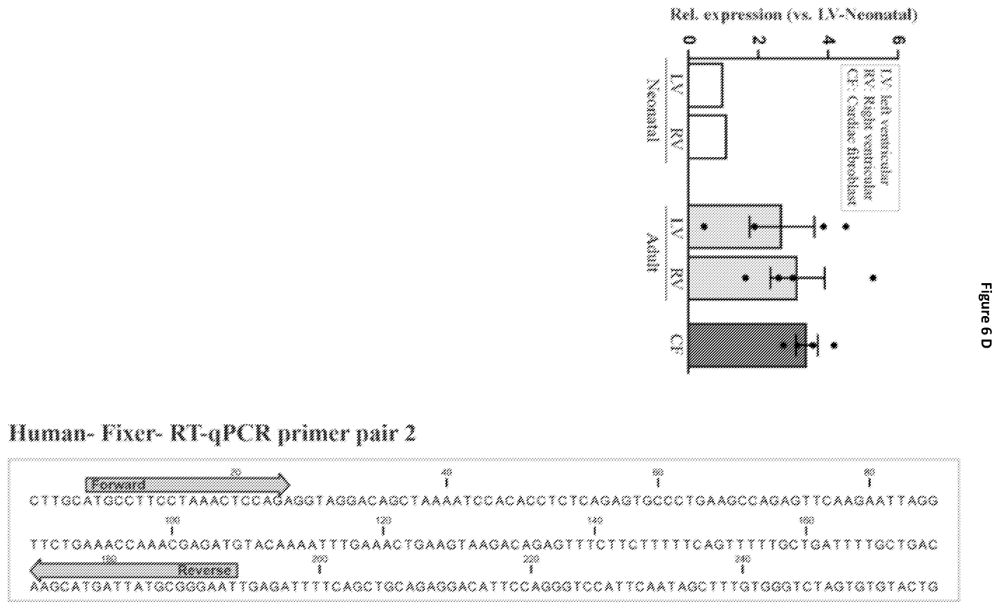

### Effects of FIXER Silencing on Cardiac Gene Expression (FIG. 5)

In order to evaluate the effects of FIXER silencing on cardiac gene expression, we performed a full transcriptomic analysis using RNA samples isolated from sham-operated and infarcted hearts, 2 weeks after MI. FIG. 5A depicts a principal component analysis (PCA) demonstrating that RNA samples isolated from infarcted hearts treated with control scrambled GapmeRs (MI-Scrambled GapmeRs) differ from samples isolated from sham-operated hearts treated with control scrambled GapmeRs as described herein(Sham-Scrambled GapmeRs). Samples isolated from sham-operated hearts treated with FIXER GapmeRs (Sham-FIXERd GapmeRs) did not differ from the sham-operated counterparts. Most importantly, RNA samples from FIXER GapmeR-treated infarcted hearts (MI-FIXER GapmeRs) are transcriptionally closer to RNA samples isolated from normal hearts, indicating that the anti-Fixer treatment produced beneficial effects.

We next analyzed the transcriptomic response. We identified 209 protein-coding genes (PCGs) uniquely differentially expressed in FIXER GapmeR-treated sham-operated hearts as compared to sham-operated hearts exposed to control scrambled GapmeRs. In contrast, a much larger set, i.e. 1734 uniquely differentially expressed PCGs were identified in FIXER GapmeR-treated infarcted hearts compared to infarcted hearts exposed to control scrambled GapmeRs (FIG. 5B). Among those, 1399 PCGs were downregulated and 335 were upregulated (FIG. 5C). Of crucial importance, the downregulated PCGs encoded primarily matrix proteins and were associated with biological pathways linked to extracellular matrix organization and collagen crosslinking (FIG. 5D). This confirmed the prominent role of FIXER as a regulator of the fibroblast gene program, and thus the large impact of anti-FIXER treatment on the development of fibrosis.

To gain insights into the global reprogramming operating in cardiac fibroblasts exposed to anti-FIXER treatment, we also evaluated the expression of lncRNAs in sham-operated and infarcted hearts receiving or not FIXER GapmeRs (FIG. 5E). Therefore, 306 lncRNAs were significantly downregulated and 374 upregulated in infarcted hearts after FIXER GapmeR administration, supporting a coordinate lncRNA-mediated response in the heart. In addition, it indicated that the lncRNA transcriptome reacted globally to the perturbation induced by FIXER knockdown. We next identified for each lncRNA its proximal PCG, i.e. the closest PCG. Given the known regulatory roles of lncRNAs on PCG expression, we performed a correlation analysis to determine whether lncRNAs could control proximal PCGs. Interestingly, expression of lncRNAs and their neighbouring coding genes were more correlated with each other than lncRNAs with randomly selected PCG genes (FIG. 5F). This further supported coordinated reprogramming of cardiac fibroblast identity via modulation of lncRNA expression and subsequent PCG expression.

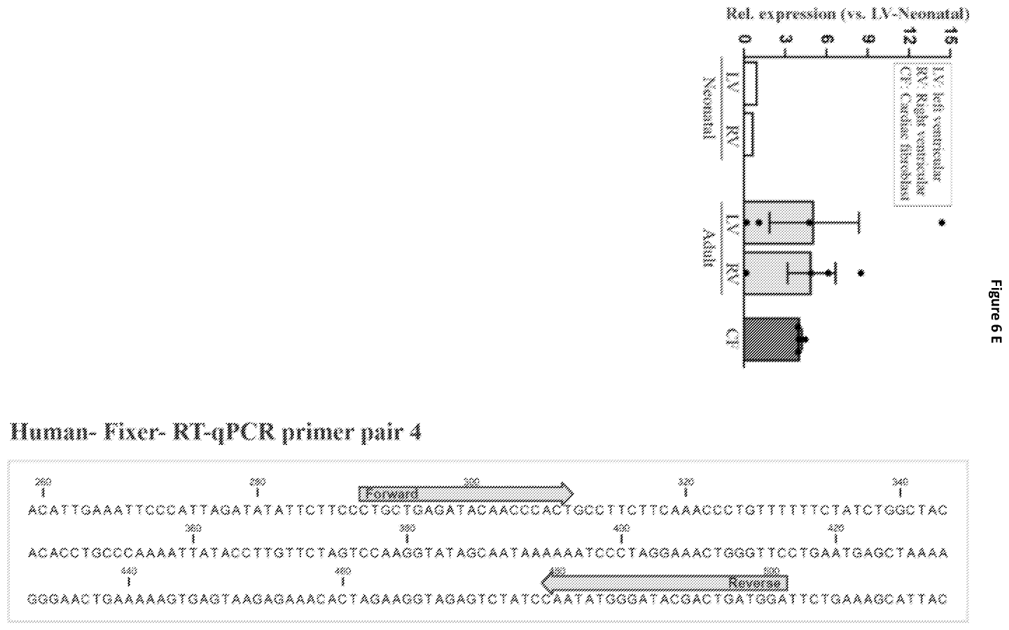

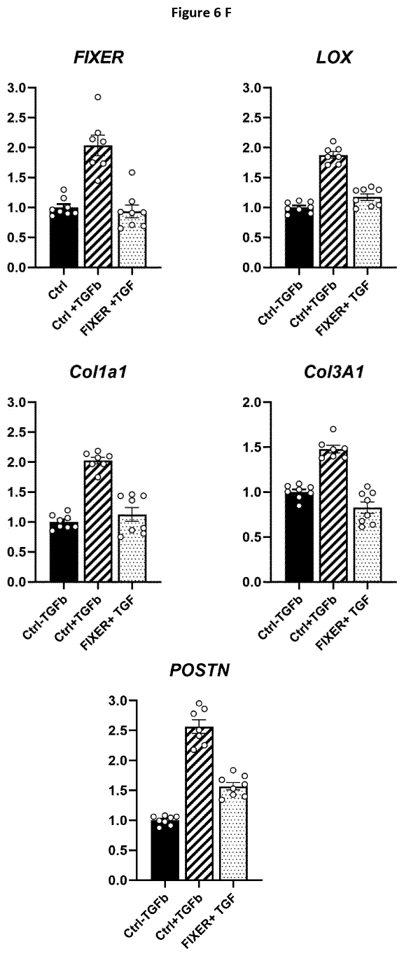

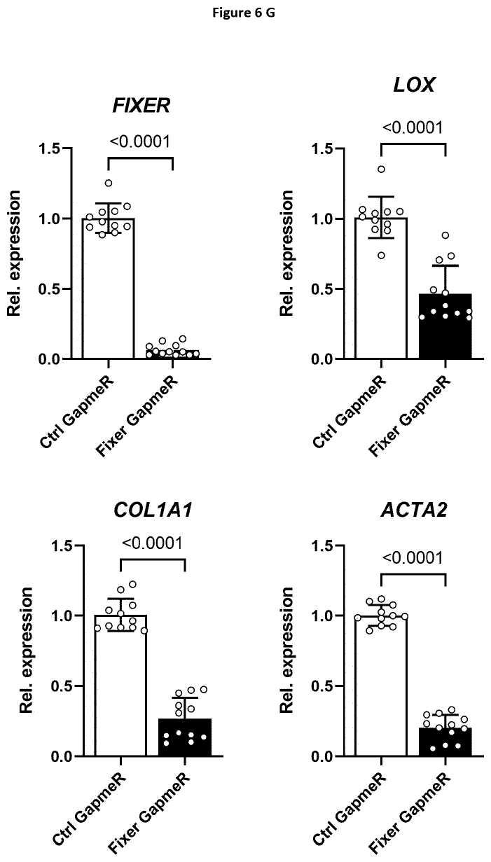

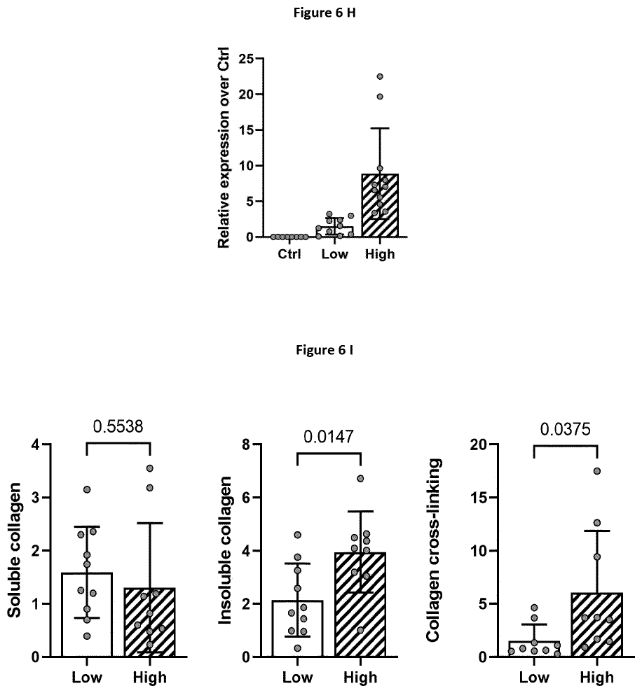

### FIXER, a New Therapeutic Target in Humans (FIG. 6)

In order to determine whether FIXER could represent a therapeutic target in human, we first evaluated whether FIXER was evolutionary conserved. Using bioinformatic tools, we detected orthologous sequences of mouse FIXER in the human genome. Importantly, these regions demonstrated positional conservation. Specifically, the predicted human FIXER is located in the human LOX locus as observed for the mouse gene. Conserved sequences were predicted to correspond to exons in the human FIXER sequence (FIG. 6A). We designed therefore primers to amplify regions corresponding to human FIXER conserved regions (FIGS. 6B and C; primer pairs 1 and 3). Using RT-PCR, and RNA samples isolated from the human heart, followed by sequencing, we were able to detect relevant amplicons, suggesting that the conserved syntenic regions in human produced an orthologous lncRNA.

We next measured FIXER expression using quantitative (q)RT-PCR (FIGS. 6D and E). Primer pairs, designed to amplify short products suited for qRT-PCR (primer pairs 2 and 4), made possible FIXER quantification in human RNA samples isolated from right and left ventricles (RV; LV) and from cultured human cardiac fibroblasts.

We also demonstrated the involvement of human FIXER in the differentiation of human CF into myofibroblasts using a knockdown approach. A GapmeR (SEQ ID No. 14) was designed to target FIXER. Thus, FIXER was silenced in human adult CFs stimulated with TGF-β after GapmeR transfection. Consistently, the expression of fibrosis marker genes was increased by TGF-β treatment and downregulated upon GapmeR-mediated FIXER knockdown. (FIG. 6F). This result demonstrated that FIXER silencing inhibited the differentiation of CF into myofibroblasts. Moreover, FIXER silencing in human cardiac fibroblasts isolated from the heart of aortic stenosis patients also reduced the expression of the fibrogenic gene program (FIG. 6G). Finally, we measured the expression of FIXER in the fibrotic heart of aortic stenosis patients. Importantly, FIXER expression correlates with the severity of the fibrotic disease, and more precisely, with cardiac insoluble collagen content and collagen crosslinking (FIGS. 6H and I).

1. A method for diagnosing a cardiac pathology in a subject, the method comprising:
a) measuring, directly or indirectly, the level of at least one transcript encoded by a DNA sequence comprising the sequence of SEQ ID NO: 1, a fragment thereof, or a variant sharing at least 80% nucleotide sequence identity thereto, in a biological sample of the subject, and
b) analyzing the level of the at least one transcript, a fragment thereof, an isoform or variant thereof, in conjunction with respective reference value ranges for the transcript,
wherein the transcript is a lncRNA, and
wherein differential expression of the lncRNA, fragment, isoform or variant thereof, in the biological sample compared to a control sample from a normal subject indicates that the subject has a cardiac pathology.

2. The method of claim 1, wherein the level of the lncRNA, fragment, isoform or variant thereof, is detected using a method comprising RNA sequencing, microarray analysis, polymerase chain reaction (PCR), reverse transcription polymerase chain reaction (RT-PCR), dual-labeled probe method, Northern blot, serial analysis of gene expression (SAGE), immunoassay, mass spectrometry, or a combination of one or more thereof.

3. The method of claim 1, wherein the cardiac pathology comprises Blood vessel disease, such as coronary artery disease; Stenosis, such as aortic stenosis; Myocardial infarction; Heart failure, such as heart failure with preserved ejection fraction, heart failure with reduced ejection fraction, and heart failure with mid-range ejection fraction; Heart rhythm problems, such as arrhythmias; Cardiac fibrosis; Heart defects, such as congenital heart defects; Heart valve disease; Genetic heart disease; Cardiomyopathy such as idiopathic and dilated cardiomyopathy; Diabetic cardiomyopathy; Toxic and drug-induced cardiomyopathy; Heart infection, such as myocarditis (incl. COVID-19); Rheumatic Heart Disease; Hypertension, such as essential and renovascular hypertension; Pulmonary hypertension; Muscular dystrophy; Cardiac pathologies associated to neurological disorders; Cardiac pathologies associated to cancer; Traumatic heart disease; and pericardial disease, affecting or not the function of the atria or the cardiac ventricles of the right or left heart, ischemic cardiomyopathies, hypertensive cardiomyopathies, diabetic cardiomyopathies, hereditary cardiomyopathies, cancer therapy-induced cardiomyopathies, ad-ageing, or a combination of one or more thereof.

4. The method of claim 1, wherein the biological sample comprises whole blood, serum, plasma, semen, saliva, tears, urine, fecal material, sweat, buccal and nasal smears, amniotic fluid, tissue sample, biopsy, hair, or a combination of one or more thereof.

5. An agent capable of modulating the expression and/or activity
i) of at least one lncRNA, a fragment thereof, an isoform thereof and a variant sharing at least 80% nucleotide sequence identity thereto, encoded by a DNA sequence comprising the sequence of SEQ ID NO: 1, a fragment thereof, or a variant sharing at least 80% nucleotide sequence identity thereto, or
ii) of a DNA sequence comprising the sequence of SEQ ID NO: 1, a fragment thereof, or a variant sharing at least 80% nucleotide sequence identity thereto, wherein the DNA sequence encodes at least one lncRNA, a fragment thereof, an isoform thereof, and a variant sharing at least 80% nucleotide sequence identity thereto.

6. (canceled)

7. The agent of claim 5, wherein the agent comprises a nucleic acid, a chemical compound, a peptide or analog thereof, an antibody or an antigen-binding fragment thereof, an antibody mimetic, or a combination of one or more thereof.

8. The agent of claim 7, wherein the nucleic acid comprises a nucleic acid encoding a siRNA, a shRNA, a miRNA, a tRNA, a piRNA, a hnRNA, a snRNA, a sgRNA used in a CRISPR-based loss- or gain-of-function system, an esiRNA, a single-stranded DNA, an antisense oligonucleotide, a fragment of one thereof, or a combination of one or more thereof.

9. The agent of claim 8, wherein the antisense oligonucleotide is an antisense oligonucleotide selectively targeting the lncRNA, fragment, isoform or variant thereof.

10. The agent of claim 9, wherein the modified antisense oligonucleotide is a GapmeR or a GapmeR with fixed chemical modification architectures.

11. The agent of claim 10, wherein the GapmeR with fixed chemical modification architectures comprises i) a GapmeR with five 2′-O-methoxyethyl (MOE) modifications in each flank, and a central gap of 10 unmodified dans (e.g. 5-10-5 MOE design), ii) a GapmeR employing three or four locked nucleic acid (LNA) modifications in each flank (e.g. 3-10-3 or 4-8-4 LNA designs), or a combination of one or more thereof.

12. A vector comprising the agent of claim 7, wherein the agent is one or more nucleic acid.

13. The vector of claim 12, wherein the vector is a gene delivery vector.

14. The vector of claim 12, wherein the vector comprises a viral vector, a non-viral vector, a particulate carrier, or a liposome.

15. A host cell comprising, or modified by the introduction of, i) one or more agent of claim 7, wherein the agent is one or more nucleic acid, or ii) a vector comprising the nucleic acid.

16. A pharmaceutical composition comprising a therapeutically effective amount of:
i) an agent of claim 5,
ii) a vector comprising the agent, wherein the agent is one or more nucleic acid, or
iii) a host cell comprising the vector, and
a pharmaceutically acceptable carriers, diluents and/or adjuvants.

17. A lncRNA, fragment, isoform or variant thereof encoded by a DNA sequence comprising the sequence of SEQ ID NO: 1, a fragment thereof, or a variant sharing at least 80% nucleotide sequence identity thereto.

18. A kit comprising:
i) a composition comprising the lncRNA modulator wherein the modulator comprises a chemical agent, a RNA mimic, an antibody, an engineered protease, or enzymatically active RNA, or
ii) a pharmaceutical composition comprising an effective amount of the lncRNA modulator wherein the modulator comprises a chemical agent, a RNA mimic, an antibody, an engineered protease, or enzymatically active RNA.

19. A diagnostic kit comprising:
i) one or more agents for detection of at least one lncRNA, a fragment thereof, an isoform thereof and a variant sharing at least 80% nucleotide sequence identity thereto, encoded by a DNA sequence comprising the sequence of SEQ ID NO: 1, a fragment thereof, or a variant sharing at least 80% nucleotide sequence identity thereto,
ii) a container for holding a biological sample isolated from a human subject, and
iii) printed instructions.

20. The diagnostic kit of claim 19, further comprising one or more control reference samples and reagents for performing an immunoassay, a Northern blot, PCR, microarray analysis, SAGE, or DNA/RNA-sequencing.

21. The diagnostic kit of claim 19, wherein the one or more agents comprise at least one probe that selectively hybridizes to the lncRNA, fragment, isoform or variant thereof, or at least one antibody that selectively binds to a lncRNA, fragment, isoform or variant thereof, or at least one set of PCR primers for amplifying a lncRNA, fragment, isoform or variant thereof comprising a sequence of SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9, or a combination of one or more thereof.

22. A method of treating a cardiac pathology in a subject in need thereof, comprising:
i) administering to the subject a therapeutically effective amount of an agent of claim 5, or
ii) administering to the subject a therapeutically effective amount of a pharmaceutical composition comprising the agent.

23. The method of claim 22, wherein the cardiac pathology comprises Blood vessel disease, such as coronary artery disease; Stenosis, such as aortic stenosis; Myocardial infarction; Heart failure, such as heart failure with preserved ejection fraction, heart failure with reduced ejection fraction, and heart failure with mid-range ejection fraction; Heart rhythm problems, such as arrhythmias; Cardiac fibrosis; Heart defects, such as congenital heart defects; Heart valve disease; Genetic heart disease; Cardiomyopathy such as idiopathic and dilated cardiomyopathy; Diabetic cardiomyopathy; Toxic and drug-induced cardiomyopathy; Heart infection, such as myocarditis (incl. COVID-19); Rheumatic Heart Disease; Hypertension, such as essential and renovascular hypertension; Pulmonary hypertension; Muscular dystrophy; Cardiac pathologies associated to neurological disorders; Cardiac pathologies associated to cancer; Traumatic heart disease; and pericardial disease, affecting or not the function of the atria or the cardiac ventricles of the right or left heart, ischemic cardiomyopathies, hypertensive cardiomyopathies, diabetic cardiomyopathies, hereditary cardiomyopathies, cancer therapy-induced cardiomyopathies, ageing, or a combination of one or more thereof.

24. The method of claim 22, wherein the agent comprises a nucleic acid, a chemical compound, a peptide or analog thereof, an antibody or an antigen-binding fragment thereof, an antibody mimetic, or a combination of one or more thereof.

25. The method of claim 24, wherein the nucleic acid comprises a nucleic acid encoding a siRNA, a shRNA, a miRNA, a tRNA, a piRNA, a hnRNA, a snRNA, a sgRNA used in a CRISPR-based loss- or gain-of-function system, an esiRNA, a single-stranded DNA, an antisense oligonucleotide, a fragment of one thereof, or a combination of one or more thereof.

26. The method of claim 25, wherein the antisense oligonucleotide is an antisense oligonucleotide selectively targeting the lncRNA, fragment, isoform or variant thereof.

27. The method of claim 26, wherein the modified antisense oligonucleotide is a GapmeR or a GapmeR with fixed chemical modification architectures.

28. The method of claim 27, wherein the GapmeR with fixed chemical modification architectures comprises i) a GapmeR with five 2′-O-methoxyethyl (MOE) modifications in each flank, and a central gap of 10 unmodified dans (e.g. 5-10-5 MOE design), ii) a GapmeR employing three or four locked nucleic acid (LNA) modifications in each flank (e.g. 3-10-3 or 4-8-4 LNA designs), or a combination of one or more thereof.

29. The agent of claim 10, wherein the GapmeR or GapmeR with fixed chemical modification architectures comprises SEQ ID NO: 12, SEQ ID NO: 13, SEQ ID NO: 14, SEQ ID NO: 15, SEQ ID NO: 16, or a fragment thereof, or a variant sharing at least 80% nucleotide sequence identity thereto.

30. The method of claim 27, wherein the GapmeR or GapmeR with fixed chemical modification architectures comprises SEQ ID NO: 12, SEQ ID NO: 13, SEQ ID NO: 14, SEQ ID NO: 15, SEQ ID NO: 16, or a fragment thereof, or a variant sharing at least 80% nucleotide sequence identity thereto.

31. The method of claim 9, wherein the antisense oligonucleotide is a modified antisense oligonucleotide.

32. The kit of claim 18, wherein the pharmaceutical composition is combined with a pharmaceutically acceptable carrier, diluent and/or adjuvant.


SEQUENCE LISTING (20 sequences)

SEQ ID NO: 1 - DNA, 19648 bp, Homo sapiens
gtaacttcattgtaaacaaaacagttttgtggtagagtaaatagatttaaggctatcccattgaaaattttcatttgtaaattgaataattcatgtaactaagcagtcttttttacatcttgttaggagcctggttattttataaagtaccctgtgttgagaatggtgtttaggtatttccacaatgactgcgtggcaaattgtgaatgagatgagaggaaatactttaagatgatctaacactaaattctaaggtttatgtggcagaatgcatgcatgagtgttgagatagggttcttcaaaatgctgtgactcagtttgggagataacagggcgccagctaaaagcagctccagctgcagtccaaatggtgatattttcatatgtgaagtgctaataagcagttagtaatctttcaagcaaaccacaaaggaagtccacaaattactacagaacactttatttatgttaacactcttgcacacaatcaaataggctttttatcctaagtgtctccattaaaggttttcagaaaaagaaaaaaaacaacaacaaaacaaaacctaagactagctataggaacagcaacaaaaaacagaaacagaagttactgttttattaacacacttgtgttatgctataaccctgtactaataatcagttatataaacaagtgattagtgagaacaaaactgacacaatgacacaattcaatcatttccttattattaatggatgttatcaagttgatatttctggaagtaaataatatattttcccattaatactaagtaactacaaaataaaatactgctataatatattacataatcacaatcatcttttttaaagatatgtttttcataatggcccccatgttaatttctgagagactagaccataacactttgtaagagaatggtgatctgttttaaatattcgatgtttatttcacattagctagagactgacactgaggattaagcctaaagacatgtttaatgaagacaggcttaagtatgtgctctctattttggcatttttcttggagactgagtgaagtttctaattgcttaattactattaggatgattgtctattgtttattgcagcggttcccaacgtttttggcactagggaccagtttcatggaaggcagtttttccatggaccaaggttagggtgtggagaatggtttcaggatgagtcaagtacattaaatttattgtgtactttatttctattattattacattgtaatatataatgaaataattatacaactcaccaaaatgtaaaatcagttgttttcctgccactgtacagtcccatctgggggtgatgggagacagtgacagaccatcaggcattagattattataaggagcatgcaacctaaatccctcatatgcacagttcacaatggggttcacgctcatatgagaatctaatgccatgattgatctgacaggaagcaaagctcaggtggtaatgtgaggaaagaggatcttggtaaacatgctattgtaagtttaatgtctctactccccttccgagaaccccatgtctccttactgggaaacttcaagtcaaatggttttaggatttactgacagaggttacttgctagtatgaatttgagaaattcatgggaggtacctcaccaggaacccaaaaaatggaatttttgacgtggggagtgtctgtaaatacagatgaagcttcacttgctggcccactgctcacctcctgctgtgcagcccagttcctaacagggatggtactggtccgtgtcctggggtttgggaaccactggtttattgaaaagacattctgctgggttggtatggagcccaagttatatttgggttacacattagggactacttcttgtgattttgagcctaaattataataaactctagatatacacaccctatgcctatcagaatagtaatgaaggggtaaatggcccccaacacaagtggcaatggtttttggaataataccaaaagccaacgaaatgtgatggagatttactgaagctaagcattaattttggggataggatgttctcgatgtaggcctagatgtatgcagcatcgtgtaaggctatctattaaagaagtctaaaaaattcacttgtgatattgagtcataaagttggtttttaatccattctcaacagactctcaatttttaaaatttttgtcaacttcttataataattcctgggagaaaagaggatcttggtaaacatgctattataagttgaatgtctctactcctcttccgagaaccccatgtctctttactgggaaacttcaagacaaatggttttaggatttagtgacagaagttacttgctagtatgaatttgagaaattcatgggaggtacctcaccaggaacccaaaaaatggaatgttttatgtggggagtgaatgacttaaaactgcattgggataaatccttggaaacagaaaaatattctctttaggtctagtgaactcaaaaccacgaaatactttcctttcacggtggacttcctcagttctctagaatgaatgtatggtaggatggagacttactggaattcatatgtcaagttcataactgagtgggtagaagctctccttttaattcctacagtaaacgacaaaaatgagtaatcctctagggggcaatagagtatgacttaggctcagaattgccgggatcaagatcctgagggaaagattggtcatgaagaattcttaaactgtcttacacttgaaaaaaacagtcggctggttcaactttatcacagcccctcaagaatctccctgattttttgtggtaaggctgaacagcaccgaggtggaacaagggatgagtgtagctgccctgccggtgcattacaaatggatggaacctgaaccccagaactggggaagatgagaagaggaagggttgcagcaaggctgactcaggctcttgaagtgggatgctctccagtgaggtttctgaccctgaggagcccgtcttgtcaggggctttactggggacggtgtgcttttatttattttcagtatgaattttcagctcttcagttctctacattttatagtacccaggacggcagaaaacttttcctaggccactttggatttaccaaaactttggtggtttggcctgcatcatgctgctttagttttgttggtgcagctcatctagcgatcaactgctcaggtaatagaaagaactccgtgaagttatcttcgtcctcatattacgaaaacaatgaccctgggtgggtggggagtaaaatcccaatgctgatatacagtcagccctccctgtttatgggttccgcatccatggattaaacaaccactgatggaggaaaatgtttaggaaaaaaaatgtgtctgtactgaatatgtagactttttttcttatttaggtagcatttacattgtattagatattataaagaatcttaaaatgatctaaagtatatggggggatatgcataggttatatgtcaatactatgccattttatatcagagagttgggtatccactgattttgttatctgtgagaggtcctggaatgagtcccccataagtaccaagagatgactatattgcctttccaaagttctagtggaaacttggactgacataggtgtggtggctttccagggtacctgttggtaagcctgcctgacctaggtgaagaagagaaaaagcatgttagagtgatgagaggagggaatgtggtgtccgaacacatacggcattttcttcttggtagagctgcatgaagaactaatgggggaaaggggtgacagaggacagagtatttttgcaaacatttatagtctctcttgcccctttgttttcttccaaggagaaaaaaattcttactggctggatttcattttccatgaagaatgtggggtgatgagatagttcgtatgagaaaaacaatcaaaacacaggaggagaataaacttgacagagggctgcgtacagcagggaaagtcactgtgggcagccggagctattgggaaagaagtggtcttagtaaaaggacagttctggaaggaagatgagagagaaagggtgccagtgattggatcattttagaggactgagttgagtttgtaaagagatgaggaataagaaaactatccagcaaagaatgttaagtcatgtggcttatattaacagtatgctgtctgttttcaggcattttccccaccaagagacttagtaaagattccaaatacttttattgactgtagaggaataggccatttgggggcagaactcaattatctttagaatacaattatctagtataacgatttcgaatgttattatctgctaacctaaattacttcttttagctctttgttcactttatatttggataatacactttcctaagtagcaataaatctgaaaaatattttagaaatgtatttttaattcaacaaaaatataaagttctagctgggtattaccaggctttccgccaagtacagtaattgttacttgtggaattttcttagaatggccaaggcatttgactcaataactataactctggaatgagtagctgggtggagtcactagtcttttggaaagttggtgggttagttcaagcctatatgctagaagaagagatttataggtggattcactcggcagatctgagaaacatagggtgtatgttttattcaaattgaacattaaatttcattacctatgcgatttgttccccctttctcctaaccaatggtttgtttcttttttctttttcttttttttttttgagacaaggtctcactgtcacccaggttggggtgcagtggtgcaatgttggctcactgcaacctccgcctccagagttcaagcgattctcctgcctcagcctcccagctagttgagattacaggctcctgccaccacatccggctaatttttgcattttttttttttttttagtagagacgaggtttcaccatgttgcccaggctggtcttgaacttctgacctcagatgatccacctgcctcagcatccctaagtgctgggactacagatgtgagccaccactccaggcccgtttgtttctttttcccttttatcttcatgcttttttaaataccatggtgataaaccatctattaaattatagtctttagaatatataaatgcaaagtccacatttaagtcaggttaggaagtgggtgggagttgtcagttatttgagaacacacatgtactgaaaacaaccacaaagctatgaggaactgaggcaaatttagggagcagagggcgacaggatcactgctcaaccaagcaacacagcaggcacagttctcagccacaatgcccagatcctttctttgagttgaaactgaatttccttgttattctttccaagatggaaatgtgtagaaccaatgtgtagaaatgcagaatataaatgcaagcaaataaattgcaaatgggacagcctaagtacaaaagttaacttttaaaaatcatttgtgaataagttggttcatctccaaagtgttacttcagatgtgcccaagtgcatagaaaaattctgacaagcttggatttgtaataaacatacttcttggatgcttcatatttccaaaatgttaataagctaaattttgagtttaaaattagcttaaacaaaatgttagcgttttgatttgaaactttatggggcatgtgtcccccccttgcatgccttcctaaactccagaggtaggacagctaaaatccacacctctcagagtgccctgaagccagagttcaagaattaggttctgaaaccaaacgagatgtacaaaatttgaaactgaagtaagacagagtttcttctttttcagtttttgctgattttgctgacaagcatgattatgcgggaattgagattttcagctgcagaggacattccagggtccattcaatagctttgtgggtctagtgtgtactgcagggtttaggaggtgcaggacatctctggagctgacagttgcaccttgcagatccctggattccagctaaagtatgtgttactggagccatagttccagggacagcttccagattcccagcttccactgaaaatgtggccgaggtagcagctcccttggtaagccagtgttacagaatcttcctgcaattatttctggagaccaggctagactccaatactccatctctttcaataattttataagaatgtaatttctcaatttaaattattttctgcttaaaatagagtgttttttgttatctgtaactgaatcttgattgataacaatgtttgagacaataggttacgtatttaacctacttgtgattgatgcaagcaaggaagtacttacctttttatttatttatttattttgaaacggagttttgctcttgttgcccaggctggagtgcaatggcatggtctcaggtcaccgcaacctccacctcctgggttcaaacaattctcctgcctcagcctcctgagtagctgggagtacaggcatgcgccaccacgcctagctaattttgtatttttagtagagacgaggtgtctccatgttggtcacgctagtctcgaactcctgacctcagttgatccgcccaccttggcctcccaaagtgctgggattataggcgtgagccactgcaccctacttacctttaaatgaggggataattgtaatttttggcagcaggtggtgaaacagtaaaatagtaatttttggcagctggtggtgtatcacctgtaatgagtgcctatagaaaacctggctttgacggactgagtgataaaggttgtggcatagcaggaggatatgatgaataaggataaactggctgctgcaaactgcgctggagagattttaaaaagaaatgggaaattcagggctttaaattatccattcaatgtggtgagagagccagggaatttttagccttaaaagaatcttctttcttgtggctgctgagctgatgtagccaaaaatcaatcacaaaatctgtccctatggcttgttgaatcacctggctggtttctgtcccacttggggtctcctctttcgctttggagcccccatccctctgtctctgcagggaggagcttctatcttctgctttctctcttctatcttgcctattaagctctctgctccttaacccactccacgtgtgtccatgtccttttatctaaaccggcatgaggacctagaaccctggtgttcctcccctcatccgagctgtatcacttgtatatgtagctttgagttcctactcatctaagcatgataaattcctagaaagagaattttttataaaaagatatgtgcacttttcatttgctagtcactgccaaattgccctccaagagaattatttgcacactcatcaacagcatacgaaagcgccgattttctgtatcctccccaactcagggttttgtcagatgttttgatcttcatcaactagataaatagaaatagatgtttcattatagctttaatttgcatttttattatgggttattttgagccttttttaactgtaaaagtaatttgtatttttgttcctgttaagtgtgatatccttcgtctcctggattgtgagtcctttccattttatttacggtagttaactgtataatggaataagctttttttttattgtcttgtatgttgaaatattgctctcccttggctatagtacttttattccaggcagaaagtaaagtttacttaacttttatgtgagtgactttatttttttatttttaattttttaatgatctttcacttttctgacaggcttaagaaagcctttcctattttaagagtgcaaaaatattctcccataatttttttctggtttccagaaatgtttgtatgtttctggtttctattgttgtatttgaaagtatggcattcttttttgtgtaaagcatgagattagaatctctttttgttttcccatccagatgacaaaacccacaattttaacaagtattcaagattgccttcttgagaaggttctcacagaggacatcttacccgttctattagccatgtgcccctgagggtccagtcattcctaagtgcctttcaagctgctccctcacagttggatttgttttattttttattacagcttgtaattgagagacactgaggcttgcacatcatgtaaaggcaggaagaccaccaggagaaacttatgtttttaccaagtctcatgctgctgtcaatctgatggggtgtagggctactcacacctgcaacattgcttcttaatgggttggtatcccatgtgggtaatacaggcatggattaccatacttaattatattagcaacagtatttttttaatgtaattcagttagtgatcaacaacaacaaaaaacccttttgtatatgtgaaggaaagttaaaataatagttattcttttaactgcctttattgtttttggttcaaaaataataagctttaaaatagttgtttaaaattatttacttatttatctatttatttattttagagagagtttcgctcttgttgcccaggctggaatgcaatggtgtgatctcagctcactgcaacttctctctcccggattcaagcaattctcctgcctcaacctcccgagtagctgagattacaggcacccaccaccatgcctggctaactttttatatttttagtaaagacagggtttcaccatgttggccaggctggtctcaaactcctgacctcaggtgatccacccgccttggccttccaaagtgccgggattacaggtgtgagccaccgcacccggcctatttttatttttaaagacaggatcttgctctgtcacccagcctggagtgcaggggcatgatcacggctcactgcagcctcaacctcctgggatcaagtgatcctcccacctcagcctccaaagtacctaggaccacaggtgcacaccaccatgcttggctaatttttaattttttttttttctgtagagatgaggtttcactatgttgcccaggctggtcttgaactcctgggctcaagcagtcctcctgccttggcttcgtaaagtgctggaattacaggcatgagccaccaggcctagcctaaaaatattttttattagtaaatatattgctttttttattttagaaatttcagtgtgttgttgagtcaataaaaaatgggctgctttctgagaccaattttcctaaaaaatcagaattatcaagtctgtctataaaccactcatgaatgcttcctgcaggttaaagtgtgagtggttttcatgtgactgctgcagccaatgctatacaagaatccatgaccatgtgaagaccatacgtggctgatcactgggaaaacaaaagtaggcattaataaaaaattaaatgggaaataatacgaggtaaaactaaactttgattgagaacttatttttgccaagcactatggtaagtgctatacatgcagtacctcatataatcaccaacccaagaaggtaggaactactattcctctgtttataaatgagaaaagagcctcaggtgaactgatttccagtgtcatacagccagaaagagcagaagcaggggctgtagcaactctgtcatgctgactgttagaaagcacgaagactggaggcgtcagaggtcagatgactcagggctgagcaagctgggatggaagtagaatgagaggacaaggagaactctgccgctcaggtgacctggaccccatctccccacagccaactatctctcttgctcccacttgtaaggcaaccccagtgtagagggcagtgctagggtggggtgtggcaggataagatgtggtagattctatctcttatggcattgggatagctcattggaaaagggcaaataggctgaatagggaaataggtgaaaaggaatgcaagatcccactattatcaccaggtaagcagcctttggaccaggcgctggctggaagaccaggtccacaagaaggcaaggagacatacagggaatgcctgcccctgcatctgcttagacacactagcactccggccacacagcagtccctacacagactcttacagacatagagatcacgctggggactcactgctacaaaccaggggagaaggaaggagccagagggagtggctaatctgagatggcttggagcccccagaattttaccatcttctattcatcttctgtatattttccccaagattcagctgctcttgaacactcctgccaaggaatgggtcaggcggtcagtaaccacatgaaaaattctgcctgtcgttttgatactgatggctctcagaaagctggcaactgtttggaaaaagtgtatgtgcttctgagattttaggtgtctaatctttaaaaaaaaaaaaagtcctagaattactaaagtcttttatatcacctggtttgaacttgataaattgcaagaaagcagttttaaaacttgtgtgagactctgagtagcacatggacagattatcagaatctacttaaacttgctgaaatctatattttatcacagattactatatattgctgtctcaagaactttattattagatcattaagtgttgtatctaacaacaagatttttacctgggtttatgagtaaatagaaaaaaatgacaatattttattaataacaagatctctgaaaatatatattatactgtgtactcagcctagtaatttaaatgtgttttatataataagaattcatagtactttcttattaggggaactaaaaattaatttgcttcgtaactcgtttctcctccaaattgaaaaatagaaaagtgggaaagcagttaccatggctatgaaaagaaggtgggatttgcatttcaatttggtgaaataatttatgcaattaagtaatgaggtttagtggagaccacatagttttagcagccagacatgccagttctgcttttgtcactgattactctgtaccgagggttagttcattaacttctctgagcctcctcagttttctgtaaaacagggataacaaaaactatcttggaaggtagttaagaggataatggcacatagcagtcgttcaataatgatattggttattattattaatgaattgaaagtgaattgtcagccaaaagcaaaatgttcttcctttgaatgggaagtagttaggttcctggactaaagttgattatagttactatcatgcaaattaaaccgtagtctgttgaattgcaggaattctttatccctataatatgttctagtaggacagaaacagtttggtgtttttggtgactttagagtgtaagatcaatgcctcgtgagtggaactattacccctgagctgaaaacactgggcaacgccctccaagcaagtcccttaaccactctcaaaaatattgcagctgggaagccacgttcatgctaaatttaaaagacatgctgattttgttttggaaaaatcaattcatcaatatgtttttttttttcatggctataaaaagtatttaaaaatcaaacaaaataaagtagtaaaaaatcctggtatgtatttttcccttggagaatgaggtactcattcaagtcctcttggaaaagattattttgtgctctggtcaaggtggcctactttccttttgtgttgaaaggatttgaaattagaagttatttgtattttcccagggagtgtccactttaggaagaaagaaaattatatgcctgaagaacatggttggccaatttattgttgtttacgtctgctaggccagaaaggaaatacatagaattcaagtttcctcttttcttctgggaaatatagtgttccattttgttttatgacagaataatattctttcctatgtttgagagtgttcaaaggtaattttttgagcatggcttgcaatctagaccaaaccacatacatgcataagaaatggaggaaagaaagtctttccttacaagatttcaataaatatgagtttctcctcttctgaaaggttgcttaaacacattttagttcctctagagcattatttttgaaaattatctacaaaaataatttttttcctctgtaattgtttcctattcgtatggacttgttcttgcaccctgtgtagaaaaatcagtatctcagccacctaggataacatattgtaggagacagtcatcaggaagaatgtgtaatcgaacttatccttagatcaaaaacagtttacagtggacacataaaataaattgatttgtaaaatgcaaatggtagagtccaaaaggaatcttaaaataaaacctggtgttgactgaattattttattcatttgtgctaggtgccctctcctggtcatagtgagtaacgtttagtagcttactctttctagattctactctgccaagaaacatgtgtgcaattgtgcatatactccatgctaggcttcatataggagggagaaaaatctgaagacaagtagcttttaactttacgagagttagactcttactttcctcctacacatagatgctttcagttatcaagcaatatttgattagcttggtgtggtggggataatactttgttaactgatatactccttttttttctttatgacttcaccatcaatccaagaaagtcctgcaaatggactgtatgtacatgacagaaatgacgtgtaaaacaagaatgcagcctagaacctgaacagaccagggtgaacatcttccttttctcctcatcctcactcccagcatagcagcattagtttatataagtcccaggagcatcaaaccccagagaaggggtggccccaacatcattgaaggttgaaaccaaaatagctgtgaaatcactgaattttgctgattttatcttaagtatttagcataaaatagaatggggtcccagaaggagaaacttattactgaaaaattgtatcttggtatatctgtttaaaaactgtgattgaggccaggcacagtggctcacacatgtaaccccagtattttgggaggctgaggcgggaggattgcttgaggctaggagtgcaaagctgtagtgagctatgatcatgccactgcacttcagcctagactacagagcaggatcctgtctcaaaaaaaaaaaagcaacaactgttattgaatgattgacttagagattctcatgtctaattcatgtctaatgcgcaacagaaaaacacagttgcagaacacagcttactgctcagcacatcagagttgggaatgactatagacgtcctctagcccagttccttgtctgggatacactgaatatttttaatctcttctgagagatgaggtaagcatattcccccagattgaaaagagaatgggcttgcttattgttttctataaaagcagtggattccccaagttcagtgttccttagctgtgatgcaaacccactgtgtgtcccaaatatctgaacacattgagtcatgctcatgggacttgagcaggaagaacctcacagcaggaagcttgctgtgttatgcttaataaactcctgtgtttctgatccaagagtctatgtcttcatatgaaatcataacaggctgacgtaatagtttgtaagtagagtaaaataaaattccagacctgacaatggtctctcacaaggttgtattcttttttactgtaccattctataattagtatttttaccttcttcagatgttagatactaggccagttccaaaaagtaattaatttatcttattgatctatcccactttgatgatgggctatttatagaaatcaataaattaaaaaatacttagaataagaaaacatgttatttctaaaaatagtaagtttcttagcagaaagcaaaactcagcaatattttcataagggatgctgtatgccttgagctgacaaacaagaaaataactaaaatgcagcatagatttgacaactgtctgttggaggggaatgaaaaaatatattttcactcagtgcagataaccctcccacactgtttctggtacaaaggaggtactcaatatttgttaaatgaattaacaaacagaaattctctaaactttattaaatctgtaatgatgaactcaaagtaatttctgtgatttccataaagaacactatcatgattaaaagacaaataatggaatggctatgaaaaagggctcattattttaagatttattttaattttaattggatacataccatcttaattatcttgtattttacttttaaaatatgaattagagacacaagtataattatggctaaagtaaaaaatagaaaatggagagaaaaaagctttgtactgtggaaaacatcaaatatacataaaaatagaataaaataatgaacccccgtttacccaacaccagcttcaataattatcaaacacagccaatctcattttatccatacttcctactcctccaactgtccgttgaattcttttgaaacaaattcatgatagcatcttagtttatctataaatacttgagtaatacacatatttttaatgaaaaaaaattcacagtaaggatggatgattgtcacaaacttaacatttctcatcaaatagaaacataaccacaaatgtcatttcacactaaaaagtgctttatgtctttggtatggtcagctttaacttcagcttaaagcagggagatcttttaaagcttaaagatccctcctccttcccatttgctttacttcgaatttgtgtcactgaatttagaatgtgttaatgacttaatgagatccaggctattaatggaaacataacaccatttcaactttaaagtagtccatcagaagtgagaatattttctgattaccaccccttttatggtaaaattattaatattggacttctagtgatggaaagtctaagaagaattagtattgtgtactaaactagtcatatttttttttacaactctttatgacttgtcagtggaaatcatttctgggagttacgttagtgtgcatatgcatttgtgtacacacacacacacacacacacaatgccttagaattataagagaattctgtagtttgttgtcacacacagagagttgtacttgctaaaatattaagatacatgaggacaaataactgctgttctgagctttgactgtgtttctgctacccaggccacctagacctctccatgatcaggcaactaaaaggttgaaatttagcacagtagagatgggtggcccacttccagcattaggcagcctccaatctgattgaaaggtgcttgtattagtcaagatctccaaagaaacagaaccaataggaggatacgtgtgtgtgtgtgtgtgtgtgtgtgtgtgtgtgtgtgtgtgtgtgtgtagagagagagagaaagagagagagagagattttttgtaaggaattagcttacgcatttatggaggctgagaagtcctgcaatctttcctctgcaagctggagacccaggagagctgggggtgtagttccagtccaagctgagggcctaaaaaacaggagaaacaatcatgtaagttcccgtctaagtgtaggagaagactgatgtcccagcttgaaaacaggttgagctagcaaactcttccttactggagacctgttgttctattcaagcctccaacggattagatgaggccaacccaccctaggatggcaaatctgctttacttaatctgatccaaatggtaatcttttctggaaactctcacaaacacactcagacataatgtttaacaaaatatctgggcatgtcatggtgcagtcaagttgacacataaaattaaccgtcacagtgtccctgctttattgattggtaaacaatttgaaaaactgcttatttaaaacatggtatagaactgatcttgggaattgataaaaagtaggcaaaactgtggcaagaaaataaggaataattgcagaaaagcagaatagaagtaactaaaacgccaggctaagagctctctttgtacattaaaaacccaggacttactcaatatctcaaagttagaggaccaggtaggaaataaatagattacaggttgaatatctctcattcaaaatgcttaggaacagaagtgtttcagatttcagatttcttcagattttggaatatttgcatatatataagatatcttgaggatgggactcctcttaacattaaattcatttgttatttatgttattcatctatgaaattcatttcccatttattatgaaattcatacaccctttattatatacatagcctgatctattgtggttttatacaatatttaaataatgtggttcatgaaacaaagtctgtattaaattcttatgtgtgaaattttccacttgagaagtcatgtcagtgctcaaaaagttttggattttggattttcaaataagggatgctcaacctttatgaagacaaaaataacaaatcacatatagagagtgccttactaagcactacaaagtaggtacatgaaaactgattgacttcgttgataaaggatgcttctggtgaactttgtaaaaaattgctgtgtaatgcaatatgaatttgctgggaatggttttgtaataaactctattaccttatgaccaattcatcaactttccgtactaaccaatattttccattccagcacactattgcccatggcttgctgaatgttcactgttggcagcttttagtctggcaagcacctaccaattgagatatatgttgaccaataccatagccagttgtgtttatttccaaatcagtttatgccagttgtacttataactttaaatatgtaaactaaacacgttatataaatcaataaactatgagtgaaaacatttaaaaagttgcttctatgaaaattaagtggaaaacttattcaaaattagattgagatgagtaactaaaaattaccaatgaattacctgtggatagttattgtaaaaaatagagtggagaatcatcttactctagaatgattttacacccaaattgcttcacaagtattttcaagagctcagtccaccttaaataagtggattaaataaaaattataaattattaataatacacaatgaggtagtttattaagaataacagcatgtgtttaatcatttgacctattggctttaaatcaaaagattggtaagcaaaagtgcattcatttgtataatttgtataatggtgaataaatgcatattttagttaaaataaaaagggaaaggtatgtataacttttataagtaagtgtttgaactgatatttaaattaatgacaactataattcctgattgtgtcaagatttctactttatgtgattattagaatcaattaaaagttaaaagttaagtattatttctgttatttttgtgtgttctttatagaactatttataagactaacagaatagaacaagagacttgtataaacctgctttgtgtctccttttcatgtacactatccatatttgaagaagagtcaaaacaaggatctaattctcttgtcctcccctttatgagagaaaaagggtgagatgacctgaaccaacaaagcatcgttatctgtatttatttattccaggagatttgtattctgcacaaggcaacaggatcattttcccagagacgggagtagccatggtgctctagactgggactcaaggagatcagtggcatttgcatagggctgaggaccaggctgtccctggagaattctgcctgagatgactgctaacacataggaatgtgtggtgcagtggttgttgcgggttggggtcgggggacctggtaaaaagtgtacagcacaattcaaatctgcaacaaaatgtagtttctttatatccagaaactgatctcatcttatcttggaaaccccattcctgctgatgatagcagaaaaaataattgcacttgccaattaaaggtaaattattctgttagttttaatttctcatcaggattaattctcagtttcaaaatttctgctccctttctttctgtccaaactggcctctggatattagtggaagcaaatgcatttttatagtatttgggagggcagtctcttcctcatgccatcctctgctcttcagcatttgctgagatgccctatctttcgtccttactgggagggatctgcagacgcctgctgcgttgtggctgttcttgtctggactggtgctatgcacttgtactgaaattcacagatcactgaccatgggcttcattgctgcctttttgtgcattaacccctgtaccacagacttccttgggcggctggtccggtacctcagtgccccacagggaagcccggaacatttgcctgcttcctccacattttgctgcagcttgcaggattctgtgagatagttcaatgcctttctctcagcgatcactattctcttcagacactaagaccaaacagaacatagggctttgctaagttcatggactggtatagcatagtctcatggactggttctgtggacacagtccattagctccaatgcccttgaattgttcctgttttgtatctttctactcacgatcacagggactttggcactaaaagaggagctaggacacttgtatatgacctcttgccaaaccttcaccctctcttggctctgctccacagcaattagaaaaaatctttttttatttctgttagatgaagtcttttcttacaccatgtcttcattcctttctttactgtggcttataatacttaggttcacactcccatgttttgatcttgatatacaaagaaaagttgggaatttcttttttctaaaagtttttgtttcaacagggtctgaattttaatgctgttgtttcacaaaatgttttaaaaatctgattttagactcaaatctgaggaatggcatttttgatccagggaatagctacacattattttcctggaataataaaactctgagtctagccttagtttttgaagagcaatatgggaaaagggaaaactggattactgtaagggaaaatatatcaaacattgaaattcccattagatatattcttccctgctgagatacaacccactgccttcttcaaaccctgttttttctatctggctacacacctgcccaaaattataccttgttctagtccaaggtatagcaataaaaaatccctaggaaactgggttcctgaatgagctaaaagggaactgaaaaagtgagtaagagaaacactagaaggtagagtctatccaatatgggatacgactgatggattctgaaagcattacctcttacataagctttcttattctaggactgggaaaacagggcagtgtggccctttatcctcagtccaaacacttcctgatcctacacatctctctcaagtgtcaaacacaaagtagcttaagaacatattctttgtctcttttgctcattaaattacagacctaaagaaaacagtgctacagatgggtgttttctcatataatatgacagccatttcactgggtcttttccaaagagaaattgacagatgcactggtgatctagcattccctcaactacaaataccatgaagccaacaagatgatgaggagcctcc

SEQ ID NO: 2 - DNA, 21 bp, Unknown
  synthetic nucleotide sequence
cttgcatgccttcctaaactc

SEQ ID NO: 3 - DNA, 27 bp, Unknown
  synthetic nucleotide sequence
aacattgttatcaatcaagattcagtt

SEQ ID NO: 4 - DNA, 20 bp, Unknown
  synthetic nucleotide sequence
catgccttcctaaactccag

SEQ ID NO: 5 - DNA, 20 bp, Unknown
  synthetic nucleotide sequence
attcccgcataatcatgctt

SEQ ID NO: 6 - DNA, 26 bp, Unknown
  synthetic nucleotide sequence
ttgatatacaaagaaaagttgggaat

SEQ ID NO: 7 - DNA, 20 bp, Unknown
  synthetic nucleotide sequence
ggaggctcctcatcatcttg

SEQ ID NO: 8 - DNA, 20 bp, Unknown
  synthetic nucleotide sequence
ctgctgagatacaacccact

SEQ ID NO: 9 - DNA, 23 bp, Unknown
  synthetic nucleotide sequence
tccatcagtcgtatcccatattg

SEQ ID NO: 10 - DNA, 19 bp, Unknown
  synthetic nucleotide sequence
gatgcagcttaagggtctc

SEQ ID NO: 11 - DNA, 19 bp, Unknown
  synthetic nucleotide sequence
ttaggttcacggtcaggtc

SEQ ID NO: 12 - DNA, 16 bp, Unknown
  synthetic nucleotide sequence
aatctcaattcccgca

SEQ ID NO: 13 - DNA, 16 bp, Unknown
  synthetic nucleotide sequence
ttacagtaatccagtt

SEQ ID NO: 14 - DNA, 16 bp, Unknown
  synthetic nucleotide sequence
ttaggaaggcatgcaa

SEQ ID NO: 15 - DNA, 16 bp, Unknown
  synthetic nucleotide sequence
aggcgattaagtatga

SEQ ID NO: 16 - DNA, 15 bp, Unknown
  synthetic nucleotide sequence
aacacgtctatacgc

SEQ ID NO: 17 - DNA, 592 bp, Homo sapiens
cttgcatgccttcctaaactccagaggtaggacagctaaaatccacacctctcagagtgccctgaagccagagttcaagaattaggttctgaaaccaaacgagatgtacaaaatttgaaactgaagtaagacagagtttcttctttttcagtttttgctgattttgctgacaagcatgattatgcgggaattgagattttcagctgcagaggacattccagggtccattcaatagctttgtgggtctagtgtgtactgcagggtttaggaggtgcaggacatctctggagctgacagttgcaccttgcagatccctggattccagctaaagtatgtgttactggagccatagttccagggacagcttccagattcccagcttccactgaaaatgtggccgaggtagcagctcccttggtaagccagtgttacagaatcttcctgcaattatttctggagaccaggctagactccaatactccatctctttcaataattttataagaatgtaatttctcaatttaaattattttctgcttaaaatagagtgttttttgttatctgtaactgaatcttgattgataacaatgtt

SEQ ID NO: 18 - DNA, 839 bp, Homo sapiens
ttgatcttgatatacaaagaaaagttgggaatttcttttttctaaaagtttttgtttcaacagggtctgaattttaatgctgttgtttcacaaaatgttttaaaaatctgattttagactcaaatctgaggaatggcatttttgatccagggaatagctacacattattttcctggaataataaaactctgagtctagccttagtttttgaagagcaatatgggaaaagggaaaactggattactgtaagggaaaatatatcaaacattgaaattcccattagatatattcttccctgctgagatacaacccactgccttcttcaaaccctgttttttctatctggctacacacctgcccaaaattataccttgttctagtccaaggtatagcaataaaaaatccctaggaaactgggttcctgaatgagctaaaagggaactgaaaaagtgagtaagagaaacactagaaggtagagtctatccaatatgggatacgactgatggattctgaaagcattacctcttacataagctttcttattctaggactgggaaaacagggcagtgtggccctttatcctcagtccaaacacttcctgatcctacacatctctctcaagtgtcaaacacaaagtagcttaagaacatattctttgtctcttttgctcattaaattacagacctaaagaaaacagtgctacagatgggtgttttctcatataatatgacagccatttcactgggtcttttccaaagagaaattgacagatgcactggtgatctagcattccctcaactacaaataccatgaagccaacaagatgatgaggagcctcc

SEQ ID NO: 19 - DNA, 258 bp, Homo sapiens
cttgcatgccttcctaaactccagaggtaggacagctaaaatccacacctctcagagtgccctgaagccagagttcaagaattaggttctgaaaccaaacgagatgtacaaaatttgaaactgaagtaagacagagtttcttctttttcagtttttgctgattttgctgacaagcatgattatgcgggaattgagattttcagctgcagaggacattccagggtccattcaatagctttgtgggtctagtgtgtactg

SEQ ID NO: 20 - DNA, 258 bp, Homo sapiens
acattgaaattcccattagatatattcttccctgctgagatacaacccactgccttcttcaaaccctgttttttctatctggctacacacctgcccaaaattataccttgttctagtccaaggtatagcaataaaaaatccctaggaaactgggttcctgaatgagctaaaagggaactgaaaaagtgagtaagagaaacactagaaggtagagtctatccaatatgggatacgactgatggattctgaaagcattac